(19) United States
(12) Patent Application Publication
(10) Pub. No.: US 2018/0042978 A1
(43) Pub. Date: Feb. 15, 2018

(54) METHOD OF TREATING PHOTO-INDUCED OCULAR FATIGUE AND ASSOCIATED REDUCTION IN SPEED OF OCULAR FOCUS

(71) Applicant: U.S. NUTRACEUTICALS, LLC d/b/a Valensa International, EUSTIS, FL (US)

(72) Inventors: JOHN A. MINATELLI, MOUNT DORA, FL (US); W. STEPHEN HILL, OCALA, FL (US)

(21) Appl. No.: 15/790,147

(22) Filed: Oct. 23, 2017

Related U.S. Application Data
(63) Continuation-in-part of application No. 14/706,092, filed on May 7, 2015.
Application No. 14/706,092 is a continuation-in-part of application No. 14/275,968, filed on May 13, 2014, now Pat. No. 9,610,313.
Application No. 14/275,968 is a continuation-in-part of application No. 13/231,131, filed on Sep. 13, 2011, now Pat. No. 8,784,904.
Application No. 13/231,131 is a continuation-in-part of application No. 12/419,321, filed on Apr. 7, 2009, now Pat. No. 8,586,104.
(60) Provisional application No. 61/043,773, filed on Apr. 10, 2008.

(51) Int. Cl. A61K 36/537; A61K 31/20; C11B 1/06; A23L 33/115; C11B 1/10; A23D 9/00; A23L 33/105

(52) U.S. Cl. CPC A61K 36/537; A23D 9/00; A61K 31/20; A23L 33/105; A23L 33/115; C11B 1/104; C11B 1/06; A61K 2236/37

(57) ABSTRACT

A method of treating photo-induced ocular fatigue and associated reduction in speed of ocular focus in humans includes administering a therapeutic amount of a dietary supplement composition comprising a carrier admixed with carotenoids, which may include 0.5 to 8.0 mg of astaxanthin, 2.0 to 15 mg of lutein and 0.2 to 12.0 mg of zeaxanthin. The composition is formulated into an oral dosage form as a single dosage capsule.

### PRIORITY APPLICATION(S)

This is a continuation-in-part of application Ser. No. 14/706,092 filed on May 7, 2015, which is a continuation-in-part of application Ser. No. 14/275,968 filed May 13, 2014 (now U.S. Pat. No. 9,610,313), which is a continuation-in-part of application Ser. No. 13/231,131 filed Sep. 13, 2011 (now U.S. Pat. No. 8,784,904), which is a continuation-in-part of application Ser. No. 12/419,321 filed Apr. 7, 2009 (now U.S. Pat. No. 8,586,104), which is based upon provisional application Ser. No. 61/043,773 filed Apr. 10, 2008, the disclosures which are hereby incorporated by reference in their entirety.

### FIELD OF THE INVENTION

This invention relates to phospholipids, seed oil extracts, and carotenoids used for eye care or other medical applications.

### BACKGROUND OF THE INVENTION

Commonly assigned U.S. Pat. No. 8,784,904, the disclosure which is hereby incorporated by reference in its entirety, discloses a perilla seed oil extract that provides a healthy source of omega-3 and contains a very favourable ratio of ALA to LA (omega-3 to omega-6) of about 6:1 in one example. This perilla seed oil extract is obtained by pressing the perilla seeds to initiate seed cracking, processing the cracked biomass using supercritical CO² fluid extraction, and collecting the extract to deliver more of the higher molecular weight compounds.

Commonly assigned U.S. Patent Publication Nos. 2013/0011469 and 2013/0295171, the disclosures which are hereby incorporated by reference in their entirety, disclose compositions and methods that enhance eye health, treat eye disorders, retard and ameliorate eye diseases and injuries, enhance other medical conditions such as cardiovascular health. The compositions include a mixture of carotenoids, including astaxanthin with phospholipid and triglyceride bound EPA and DHA derived from krill oil, in which the krill oil contains at least 30% total phospholipids. This eye are composition is administered in a therapeutically effective amount to prevent, retard or treat eye and central nervous system diseases or injuries, such as age-related macular degeneration, cataract, dry eye syndrome due to glandular inflammation and other central nervous system degenerative diseases, photic injury, ischemic diseases, and inflammatory diseases, including related to the cardiovascular system.

### SUMMARY OF THE INVENTION

A method of treating photo-induced ocular fatigue and associated reduction in speed of ocular focus in humans comprises administering a therapeutic amount of a dietary supplement composition comprising a carrier admixed with carotenoids consisting of 0.5 to 8.0 mg of astaxanthin, 2.0 to 15 mg of lutein and 0.2 to 12.0 mg of zeaxanthin, and wherein the composition is formulated into an oral dosage form as a single dosage capsule.

The carrier may comprise at least one of a lipid and a fatty acid and the lipid may comprise phospholipids that are 5% to 60% by weight of the composition. The phospholipids may comprise seed based phospholipids and the phospholipids may comprise phospholipids derived from a marine source. The composition may further include a seed oil containing phospholipid bound EPA and DHA. The fatty acid may comprise an omega-3 fatty acid and the carotenoids may consist of 4.0 to 6.0 mg of astaxanthin derived from Haematococcus pluvialis (Hp), 10 to 12 mg of lutein and 1.0 to 2.5 mg of zeaxanthin.

In another example, a method of treating photo-induced ocular fatigue and associated reduction in speed of ocular focus in humans comprises administering a therapeutic amount of a dietary supplement composition comprising 50 to 700 mg of a carrier, including at least one of a lipid and a fatty acid and admixed with carotenoids comprising 4.0 to 6.0 mg of astaxanthin derived from Haematococcus pluvialis (Hp), 10 to 12 mg of lutein and 1.0 to 2.5 mg of zeaxanthin. The composition is formulated into an oral dosage form as a single dosage capsule.

A method of treating photo-induced ocular fatigue and associated reduction in speed of ocular focus in humans comprises administering a therapeutic amount of a carrier, including a dietary supplement composition comprising 50 to 700 mg of at least one of a lipid and a fatty acid, and admixed with astaxanthin, lutein and trans-zeaxanthin, wherein the dietary supplement composition is formulated into a single dosage capsule. The astaxanthin is about 0.1 to 16 percent by weight of the at least one of the lipid and fatty acid, the lutein is about 0.4 to 30 percent by weight of the at least one of the lipid and fatty acid, and the trans-zeaxanthin is about 0.04 to 24 percent by weight of the carrier, including at least one of the lipid and fatty acid.

### DESCRIPTION OF THE DRAWINGS

Other objects, features and advantages of the present invention will become apparent from the detailed description of the invention which follows, when considered in light of the accompanying drawings in which:

FIG. 1 is a flowchart illustrating a production diagram as a flowchart for producing omega-3 chia seed oil such as sold under the tradename Chia Gold™ by Valensa International of Eustis, Fla.

FIG. 2 is another production diagram as a flowchart showing basic steps for manufacturing a perilla seed oil extract in accordance with a non-limiting example.

FIG. 3 is a chart showing the comparative Rancimat performance of various oils, including the oils extracted from chia seed and perilla seed in accordance with a non-limiting example.

FIG. 4 is a graph showing the normalized absorbance, for example, carotenoids, including astaxanthin, β-carotene and lutein.

### DETAILED DESCRIPTION

The present invention will now be described more fully hereinafter. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The seed oil extract and added phospholipids, such as from roe extract, and mixed carotenoids, including astaxanthin, such as S, S′-astaxanthin derived from Haematococcus pluvialis, and one or more of lutein and/or trans-zeaxanthin or meso-zeaxanthin, provide, in one example, an eye health care composition and method that retards and ameliorates eye diseases and injuries and prevents, retards or treats eye and central nervous system diseases or injuries, including age-related macular degeneration, cataract, dry eye syndrome due to glandular inflammation and other central nervous system degenerative diseases, photic injury, ischemic diseases, and inflammatory diseases, including cardiovascular. The phospholipids will enhance absorption of the carotenoids and other ingredients, for example, lutein alone and coenzyme Q10.

It should be understood that phospholipids can be used alone or in combination with the seed oil extract such as a perilla seed extract produced by CO2 supercritical fluid extraction. For example, the phospholipid may be formed as a phospholipid rich egg roe extract comprising at least 50% phospholipids. Often this extract will not flow adequately depending on end use and it will have added the seed oil extract having an ALA to LA ratio between 1:1 and 6:1, which may also include different phospholipids as well as some EPA and DHA in an example. Different phospholipids may be used, including at least one of Phosphatidylcholine, Phosphatidylethanolamine, Phosphatidylserine, Phosphatidylinositol, Phosphatidic acid, Lyso-Phosphatidylcholine, Lyso-Phosphatidylethanolamine, and Lyso-Phosphatidylserine. This phospholipid may be derived from at least one of a plant, algae and animal source or synthetic derivative and can include a mixture of antioxidants that are admixed with the phospholipid alone or with the seed oil extract alone or admixed with the phospholipid and seed oil extract. Phospholipids may be obtained from a marine based source such as krill oil or a plant based source such as soybean, safflower and sunflower as non-limiting examples. Another example is phospholipids from egg yolk.

The phospholipids may have no phospholipid bound EPA or DHA in some examples, and in other examples, the phospholipids may include some EPA or DHA. For example, krill oil may have in a sample 70-190 mg/g of EPA and 30-160 mg/g of DHA with the total EPA/DHA of 100-350 mg/g while fish oil is about 500-700 mg/g with the fish oil triglycerides containing a much greater content of EPA and DHA. Krill oil, algae oil, and other similar oils all have omega-3's while the plant based phospholipids typically have little or none, but include the ALA and LA. For that reason, it is preferred to use a phospholipid, such as the egg roe extract with a seed based or seed oil extract having ALA and LA in a ratio between 1:1 and 6:1 in a non-limiting example.

Phospholipids may include glycophospholipids and lyso-phospholipids. The phospholipids are used to deliver small amounts of active ingredients and include in an example the plant based phospholipids and commercially available lecithins and egg yolk and seed based oils. Phospholipids increase the bioavailability of the added substrate. One example includes phospholipids derived from vegetable sources, which usually do not contain long-chain n-3 polyunsaturated fatty acids (PUFA's). Although the phospholipids are explained as aiding the absorption of a carotenoid such as astaxanthin and lutein, it should be understood that the phospholipids may increase absorption of other components such as coenzyme Q10.

The various incorporated by reference patents and parent and grandparent applications described below disclose the use of seed oil extracts and some phospholipids. The description will proceed with the description of a seed oil extract such as derived from chia followed by a description of the seed oil extract derived from perilla and then with examples of other seed oil extracts followed by examples of phospholipids that may be used alone or in combination with seed oil extracts and carotenoids such as astaxanthin and lutein and other components such as coenzyme Q10. Some of the phospholipids are derived from plant and seed oil extracts in an example since they have the ALA and LA and phospholipids would be substantially devoid of EPA and DHA in some examples.

There now follows a description as directed to forming a seed oil extract such as supercritical fluid CO2 solvent extracted oil from respective pre-milled salvia hispanica L. seed. This description is followed by a description of forming a perilla seed oil extract. The perilla seed oil extract may be used in the current composition and method. The chia manufacturing is explained since many parts of the manufacturing process are similar to the production of the perilla seed oil extract. In another example, other seed oil extracts are disclosed.

Commonly assigned U.S. Pat. No. 8,586,104 discloses a room temperature, shelf stable mixture of an approximate 3.1:1 to about 3.3:1 mixture of alpha-linolenic acid (“ALA”, “Omega-3 polyunsaturated fatty acid” (“PUFA”)) to linoleic acid (“LA”, “Omega-6 PUFA) that has been prepared in the presence of limited amounts of saturated and mono-unsaturated fatty acids as their mixed triglycerides by the use of either supercritical fluid CO² solvent extraction of premilled Salvia hispanica L. seed alone, and more particularly, supercritical fluid CO² solvent extraction in the presence of mixtures of hydrophilic and lipophilic antioxidants, or, by the use of a common organic solvent extraction such as hexane or by the use of expeller pressing techniques. The supercritical CO² extraction is preferred.

Omega-3 and Omega-6 PUFAs are well known as essential fatty acids in man and many animals, which are useful in humans and animals in promoting, for example, a heart healthy condition in man. It is also well known, however, that PUFAs are extremely susceptible to rapid, uncontrollable free radical mediated degradation.

The chia seed composition of matter includes a supercritical CO² Salvia hispanica L. derived seed oil comprising from 60-88% PUFAs in a ratio of from about 3.1:1-3.3:1 of alpha-linolenic acid (ALA) to linoleic acid (LA), 4-10% of C-18 mono-unsaturated fatty acid, 1-5% of C-18 saturated fatty acid and 4-8% of C-16 saturated fatty acid in a mixed triglyceride form that is stable at room temperature for 12-24 months and comprising a mixture of selected antioxidants.

This composition of matter as the mixture of a phospholipid and seed oil extract may include dietary supplement ingredients, such as Docosahexaenoic Acid (DHA) and/or Eicosapentaenoic Acid (EPA) in pectin or gelatin based confectionery dietary supplement delivery systems. EPA, DHA, docosahexaenoic acid (DPA) or gamma-linolenic acid (GLA), fish oil, krill oil, krill oil concentrate, borage oil, evening primrose oil, olive oil or other plant, animal or algal based seed or fruit oils may be admixed therein either alone or in combination. Lipophilic antioxidants may be added either alone or in combination with the mixture of phospholipids and seed oil extract and with at least one of a) phenolic antioxidants including at least one of sage, oregano, and rosemary; b) tocopherol, c) tocotrienol(s), d) carotenoids including at least one of astaxanthin, lutein, and zeaxanthin; e) ascorbylacetate; f) ascorbylpalmitate g) Butylated hydroxytoluene (BHT); h) Docosapentaenoic Acid (BHA) and i) Tertiary Butyl hydroquinone (TBHQ). As disclosed, a hydrophilic antioxidant or sequesterant includes hydrophilic phenolic antioxidants including at least one of grape seed extract, tea extracts, ascorbic acid, citric acid, tartaric acid, and malic acid. These ingredients may be included with the phospholipid and seed oil extract, such as perilla seed oil extract as used for the current composition for eye or cardiovascular treatment.

In this example for chia, PUFA rich seed oil extract is prepared from Salvia hispanica L. seed which contains one of nature's more favorable seed based concentrations and ratios for the essential fatty acids, and more specifically, the essential fatty acids ALA and LA in a ratio of approximately 3.3:1 as a mixture of ALA and LA that is stable at room temperature for long periods of time when desirably and appropriately treated with antioxidants either before, during, or after (or any combination thereof). A shelf life of 12-24 months has been found.

Such oils, including the chia, and more particularly, the more advantageous perilla or other seed oil extracts, are used either alone or advantageously in combination with other ingredients, for example, phospholipids and also algae, plant or fish oil derived alpha-linolenic acid (ALA) or linoleic acid (LA) metabolites such as eicosapentaenoic acid (EPA), docosapentaenoic acid (DPA), gamma-linolenic acid (GLA) or docosahexaenoic acid (DHA) or any combination thereof, incorporated into appropriate foods, beverages or dietary supplements for the prevention or mitigation of such diseases as cardiovascular disease, arthritis, pain, blood clotting, dry eyes and brain health.

Such disease mitigation has been associated with the competitive control of the LA metabolic cascade and the resulting metabolic cascade products from LA metabolism known commonly as eicosanoids, such as the series 2 and 3 prostaglandins and thromboxanes, the series 4 leucotrienes and lipoxins and the series 5 leucotrienes all of which are potent platelet aggregators and/or inhibitors, pro-inflammatories, vasodilators, bronchoconstrictors, or anti-asthmatics and the like.

The consumption of ALA has been shown to be a very effective competitive substrate of delta-6 desaturase, which is known to be the rate limiting enzymatic step in both ALA and LA metabolism to the metabolic products discussed above.

Attempted extraction of Saliva hispanica L. (and in some cases perilla and other seeds) unmilled seed, using supercritical CO² even at extraordinarily high pressures of 1000 bar or hexane solvent at atmospheric pressures, yields very little, if any, seed oil, therefore the seed is milled prior to extraction. The extent of the milling, as measured by particle size distribution, is advantageous to the extraction process in accordance with a non-limiting aspect since the higher the surface area, the higher will be the efficiency and completeness of the extraction process by either organic solvent based or supercritical fluid based processes. In addition, it is often advantageous to mill the seed, such as chia, perilla, or other seed in a blanket of inert gas such as nitrogen to prevent per-oxidative processes from taking place that would otherwise be initiated in the presence of air or oxygen and light. It should be understood that manufacturing steps as applied to chic may apply with modification to perilla seed also, but different pressures, temperatures, cycle times, and other parameters would change not only for the different seeds, but also the desired final oil as extracted.

In one example, Salvia Hispanica L. whole seed is either first commutated in a standard knife or hammer mill or more preferably roller milled, preferably under a cold nitrogen atmosphere, to produce a cracked seed biomass. The seed biomass is preferably treated with one or more hydrophilic and/or lipophilic antioxidants by mixing the antioxidants to the resulting biomass. In another embodiment, the antioxidant may be advantageously added to the seed prior to or during the milling process or at the point of extraction without pre-blending the antioxidants evenly throughout the resulting biomass due to the nature of the extraction process. The biomass is then transferred to a supercritical fluid extraction unit for separation of the seed oil from the cracked or flake-rolled biomass.

Alternatively, the pre-prepared biomass can be transferred to a common hexane solvent extractor, or an expeller press for example, and the oil extracted from the biomass accordingly. Preferably either process is conducted in the absence of oxygen or air.

The supercritical fluid extraction of the milled seed admixed with hydrophilic and/or lipophilic antioxidants is accomplished by subjecting the premilled cracked or flake-rolled seed to supercritical CO², or CO² and propane as a co-solvent, or supercritical propane alone at from 40-1000 bar at from 30-100 Deg. C. More preferably the seed oil is extracted from the biomass between 50-800 bar at 50-90 deg. C. in such CO² amounts measured in kgs/kg of biomass and for such times as may be required to extract large portions of the seed oil content from the biomass. In addition, entrainment solvents can be added to the supercritical fluid to further enhance the efficacy of such extractions. For example, supercritical carbon dioxide extraction of the biomass can be enhanced by the addition of propane to the supercritical extraction fluid.

The resulting seed oil dissolved in supercritical solvent(s) is next allowed to fractionate in two separate pressure step-down stages allowing the collection of a light and heavy fraction of seed oil extract. This light fraction also contains water that has been co-extracted from the seed mass. The resulting seed oil, after degassing, is separated from any water that may have been carried over during the extraction of the biomass containing the water. The light fraction of the seed oil extract is rich in taste and odor components and may be admixed with the heavy fraction or may be discarded depending on the desired product characteristics.

After separation of the water remaining in each fraction, the fractions are then held under nitrogen or other inert gas and additional amounts of lipophilic and/or hydrophilic antioxidants may then be added. In addition, the resulting fractions may also be treated with bleaching clay, actuated carbon and such other processing aids as may be required to render the oil suitable for its intended use in humans and animals.

The PV of the resulting seed oil extract is typically under 2.0 meq/Km, while accelerated decomposition, using a Rancimat instrument, remarkably indicates an extrapolated room temperature shelf life of from about 1-2 years. When the same process is repeated without the use of antioxidants, the resulting PV is surprisingly under 10.0 meq/Kg most probably due to the use of supercritical CO² resulting in minimal exposure of the oil to oxygen species. However, the resulting oil quickly begins to build peroxide value in the presence of air even when stored at temperatures of 0 Degrees C. In addition, such unstabilized oils, under accelerated rancimat testing exhibit very poor stability to heat and oxygen unlike the rancimat performance observed in stabilized oils derived from the process described above. Antioxidants as described before are thus used.

The resulting supercritical fluid seed oil extract from chic contains from 60-88% PUFAs in a ratio of from 3.1:1-3.3:1 of ALA:LA, 4-10% of C18 mono-unsaturated fatty acid, 1-5% of C-18 saturated fatty acid and 4-8% C-16 saturated fatty acid composition in a mixed triglyceride form depending on the seed source employed.

On the other hand, if the process described above is conducted without the use of hydrophilic and/or lipophilic antioxidants, the resulting seed oil extract exhibits an initial low PV but accelerated stability testing using a Rancimat instrument indicates an extrapolated room temperature shelf stability of less than two months.

The stability of the resulting oil at room temperature that is manufactured without the use of added antioxidants cannot be easily explained because of the available levels of the powerful natural antioxidants found in Salvia hispanica L. whole seed whose activity can be easily measured in Oxygen Radical Absorbance Capacity (ORAC) units. Salvia hispanica L. has a measured ORAC number of 3000 micromoles TB ORAC units/gram of seed and is known to contain such antioxidants as myricetin, quercetin, kaempferol, caffeic acid, and chlorogenic acid. In addition, it is well known that the Salvia hispanica L. whole seed, unlike many other seeds bearing PUFA containing oil, exhibits a shelf life of at least 5 years due to its structure and the naturally occurring antioxidants available in the seed matrix.

In addition, cold pressing of Salvia hispanica L. whole seed also produces unstable seed oil without careful addition of appropriate antioxidants to the seed prior to the expeller pressing process.

Any resulting seed oil fractions can be treated with additional antioxidants to afford a desired room temperature stability. The extent of oil extraction can be controlled by particle size distribution of the milled or flaked seed. Propane can be added in mixture with supercritical CO² in the supercritical state as an extraction solvent. In yet another aspect solvent can be extracted using hexane extraction at or near atmospheric pressures and the resulting boiling point of hexane in the absence of oxygen, separating the resulting water from the oil/hexane mixture and removing the hexane solvent by distillation at or below atmospheric pressure in the absence of oxygen.

In one aspect, an emulsifying agent is added. In another aspect, nano- and/or micro-particles of rice or sugarcane based polycosanol are dispersed for enhancing a heart healthy dietary supplement. A stabilized oil in a fruit juice concentrate, fruit puree or other water based flavoring is dispersed in the presence of maltodextrin, or other carbohydrates, and a suitable emulsifying or emulsion stabilization agent that is vacuum spray dried to form an amorphous or crystalline solid suitable for use as a flavoring ingredient carrying stabilized PUFAs in flavored dietary supplements, foods and beverages. In yet another aspect, oil based flavors and fragrances suitable for use as an ingredient in foods, beverages and cosmetics are added. ALA and LA are also added as essential fatty acids.

The process as described relative to the chia seed oil extract may be applied to many different seed oil extracts with different CO2 extraction parameters changed for the specific seed. It is well known in the pharmaceutical, nutraceutical, and food industries that the selected processing parameters and manufacturing techniques used with CO2 extraction changes the composition of the final product, both the extracted product, usually in oil, and the solid residue, which could be left over in the extractor unit after oil extraction and be commercially sold as a flour with specific limitations placed on its characteristics. Although perilla seed is from the same mint family as chia seed, the oil extracted from the CO2 extractor and the flour left over as the residue in the extractor are substantially different. In the CO2 extraction, the supercritical CO2 has a high solvating capacity that is a function of density. By changing the density (with pressure), the CO2 extractor is able to select the quality, quantity and specific physiochemical characteristics of the oil that is extracted and any leftover residue after oil extraction as a flour.

Thus, the extracted oil from perilla seed or other seed and the output and as a left over residue remaining after oil extraction from the supercritical CO2 extraction depends in large part on the change in density, pressure and other extraction parameters. The extracted oil and any final residue as a flour can be substantially different from any other type of extracted oil and final residue based on extraction parameters that are chosen. The final output from the extractor such as the oil and any residue remaining after oil extraction also depends on the type of pre-milling used to form the cracked biomass. The type of milling or roller press flaking of the seed to obtain the cracked seed biomass can be varied. The extent and type of seed cracking is relevant to the final extraction output as the oil extracted and left over residue during the supercritical fluid extraction process.

As noted before, it has also been found that the use of a perilla seed oil extract is more advantageous and contains a very favorable ratio of ALA to LA (omega-3 to omega-6) of as high as about 6:1 in some examples as compared to chia seed oil that is typically about 3.3:1 ALA to LA. Perilla seed oil extract as obtained, in accordance with a non-limiting example, in a similar manner as described relative to the preparation and extraction of the chia seed oil. It has been found, however, that the perilla seed oil extract is an even healthier source of omega-3 than chia seed oil in some non-limiting examples and is excellent with astaxanthin and other carotenoids, including phospholipids for eye health care and cardiovascular when used with coenzyme Q10.

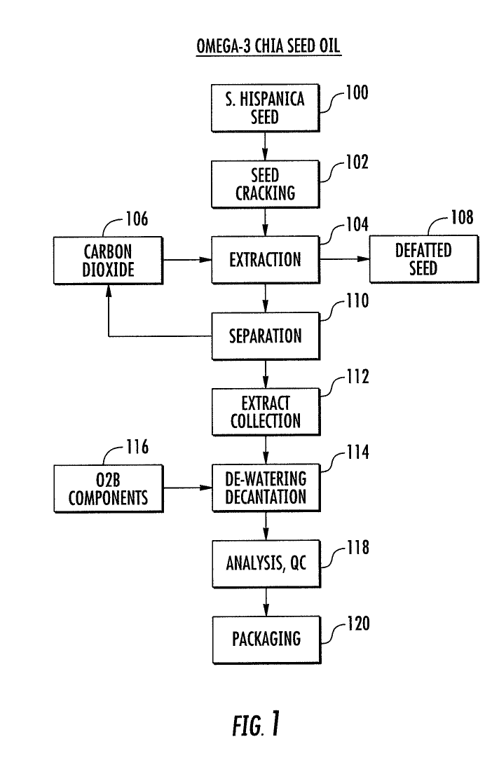

FIG. 1 is a flowchart showing a production diagram for a sequence of steps for producing omega-3 chia seed oil such as sold under the tradename Chia Gold™ by Valensa International of Eustis, Fla. The salvia hispanica seed is provided (block 100) and seed cracking occurs (block 102) to form a cracked biomass. Various techniques for seed cracking and forming the biomass can be used as described above. The supercritical CO² extraction (block 104) uses ultra high pressure carbon dioxide extraction technology and supplied CO² (block 106) such as the DEEP EXTRACT® manufacturing process developed by Valensa International of Eustis, Fla. The defatted seed as a flour, for example, (block 108) is produced. Separation of the different portions occurs such as by fractionating the seed oil extract (block 110) as described above. The extract is collected (block 112). Dewatering and decantation occurs (block 114) and antioxidants are added (block 116) such as the OTB® Per Oxidation blocker system from Valensa International. Quality control analysis occurs (block 118) and the final oil packaged (block 120).

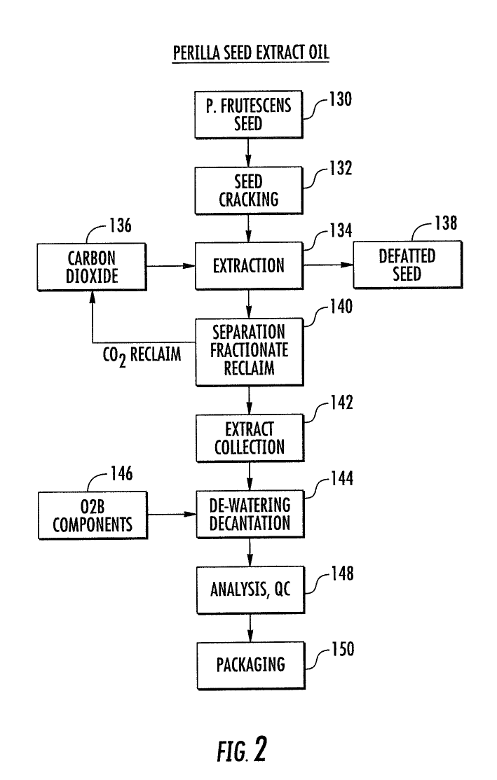

FIG. 2 shows a second production diagram as a flowchart for producing the perilla seed oil extract in accordance with a non-limiting example. This process may be used with other seeds. The process begins with a source of the perilla seed also known as perilla frutescens seed (block 130). Similar to the process with the omega-3 chia seed oil, seed cracking occurs (block 132) to form a cracked seed biomass followed by the extraction (block 134) using the supercritical CO² extraction and supplying carbon dioxide (block 136) to produce the defatted seed (block 138) that is partially or wholly defatted perilla seed as a cake residue with virtually no fat or oil. The other portion is the oil is separated and the CO² is reclaimed (block 140). Similar to the omega-3 chia seed oil, the extract is collected (block 142) and dewatering and decantation occurs (block 144). Antioxidants such as the OTB® components as described before are added (block 146) and the quality control analysis occurs (block 148) followed by packaging (block 150).

It should be understood that the perilla seed oil extract can be supercritically extracted in a similar process as used with the chia seed as described above. For example, the resulting seed oil with the perilla seed extract is allowed to fractionate after supercritical solvent extraction in two separate pressure step-down stages allowing the collection of a light and heavy fraction of seed oil extract. The process and temperatures may be modified to suit perilla or other seed oils, such as extraction from the biomass between 50-800 bar at 50-90 degrees C. and such CO2 amounts measured in KGS/KG of biomass may be used as also described above, but different temperatures may be used depending on the desired end product oil. It is possible to use propane as a co-solvent or supercritical propane alone at from 40-1,000 bar from 30-100 degrees C.

As noted above, the perilla seed oil extract can have a range of values for its fatty acid profile depending on the quality and type of seed cracking and extraction parameters. Total fatty acids, peroxide values and other component values are described in Table 1 below showing an analysis of perilla seed oil extract and various parameters, specifications and results as a non-limiting example. Results can vary of course for different samples.

TABLE 1
Ingredients:
Perilla (Perilla frutescens (L.) Britton) Seed Oil, O2B [symbol] Botanical peroxidation blocker
including refined nonGMO soybean mixed tocopherols and spice extract.

| Parameter | Specification | | Result |
|---|---|---|---|
| Description | Clear yellow oil, pourable at room temperature | | Conforms |
| Odor | Mild | | Conforms |
| Solubility | Insoluble in water, miscible with oils | | Conforms |
| Fatty Acid Prone (%) | | VQP-050 (GC) | |
| Palmitic Acid | | | 6.3 |
| Stearic Acid | | | 1.9 |
| Oleic Acid | | | 22.3 |
| Linoleic Acid | | | 9.7 |
| α-Linolenic Acid | >56 | | 59.8 |
| Total fatty Acids (% w/w) | 85 . . . 95 | | 88.6 |
| Peroxide Value (meqO₂/kg) | <10 | VQP-049 | 2.3 |
| Water Content (%) | <1.5 | VQP-048 | <0.2 |
| Heavy Metals (ppm) | <10 | ICP-MS | <1 |
| Microbiological Data (cfu/g) | | | |
| Total Aerobic Microbial Count | <10³ | USP <61> | <100 |
| Combined Yeast & Mold | <10² | USP <61> | <100 |
| E. coli/Total Coliforms | <10 | AOAC 991.14 | <10 |

(all values as is basis)
All of the ingredients are GMO free. Therefore this product is in accordance with EU regulations 1830/2003 and 1829/2003. The Product has not been treated with gamma rays. [symbol]
indicates data missing or illegible when filed

Table 2 show accelerated stability testing of the perilla seed extract with an OTB® per oxidation blocker and Table 3 shows the accelerated stability testing of a perilla seed extract sample produced by Valensa International of Eustis, Fla. as compared to chia seed extracts such as the Chia Gold™ which is produced by the process shown in FIG. 1.

TABLE 2
Accelerated Stability Testing of Valensa Perilla Seed Extract with O2B ®

| | Rancidity | |
|---|---|---|
| | Induction Time | Shelf Life @20° C. |
| | (hr)¹ | (yr)² |
| Perilla Oil (without O2B) | 7 | 0.5 |
| Perilla Oil (with O2B) | 42 | 2.8 |

¹Rancimat induction time measured in hours with air bubbling through heated oil (90° C.) in the light.
²Rancimat data is a function of accelerated high heat and oxygen exposure. Extrapolated data plots are assumed linear in the presence of air and light at 20° C. however, if product is stored at 20° C. in an air and light barrier package, then shelf stability is at least doubled.

TABLE 3
Accelerated Stability Testing of Valensa Perilla Seed and
Chia Seed Extracts with O2B ®

| | Rancidity | | |
|---|---|---|---|
| | Induction Time | Shelf Life @20° C. | Shelf Life Increase |
| | (hr)¹ | (yr)² | (%) |
| Perilla Oil (without O2B) | 7 | 0.5 | |
| Perilla Oil (with O2B) | 42 | 2.8 | 611% |
| Tresalbio Chia Oil (without O2B) | 12 | 0.4 | |
| Tresalbio Chia Oil (with O2B) | 73 | 2.2 | 603% |
| ChiaGold Oil (without O2B) | 9 | 0.3 | |
| ChiaGold Oil (with O2B) | 60 | 1.8 | 663% |

¹Rancimat induction time measured in hours with air bubbling through heated oil (80-90° C.) in the light.
²Rancimat data is a function of accelerated high heat and oxygen exposure. Extrapolated data plots are assumed linear in the presence of air and light at 20° C. however, if product is stored at 20° C. in an air and light barrier package, then shelf stability is at least doubled.

Table 1 illustrates various values and shows the total fatty acids (% w/w) is 85 to about 95 and has in that particular example a result of 88.6. It should be understood that the seed oil could possibly have as low as about 60% w/w of PUFAs and as high as about 95% and a ratio of from about 4:1 to about 6:1 ALA to LA. The peroxide value of the seed oil is typically under 10.0 meq/Km. The PUFAs typically comprise at least greater than 50% ALA and in the example shown in Table 1 is greater than 56% and in one particular example shown in FIG. 1 is 59.8. The seed oil is shelf stable at room temperature up to 32 months in a particular example. Other data is shown such as specific components of the fatty acids and the water content, heavy metals in PPM, and microbiological data in CFU/G, such as the total aerobic microbial count, a combined yeast and mold and E. coli/total coliforms. All the values are an as-is basis and the ingredients are GMO free. Therefore, this product is in accordance with EU regulations 1830/2003 and 1829/2003. This product had not been treated with gamma rays. Ingredients include the perilla (perilla frutescens (L) Britton) seed oil, OTB® botanical per oxidation blocker including refined non-GMO soybean mixed tocopherols and spice extracts. GMO corresponds to genetically modified organisms, and thus, non-GMO refers to non-genetically modified organisms. The soybean had not been created through gene-splicing techniques of biotechnology or genetic engineering.

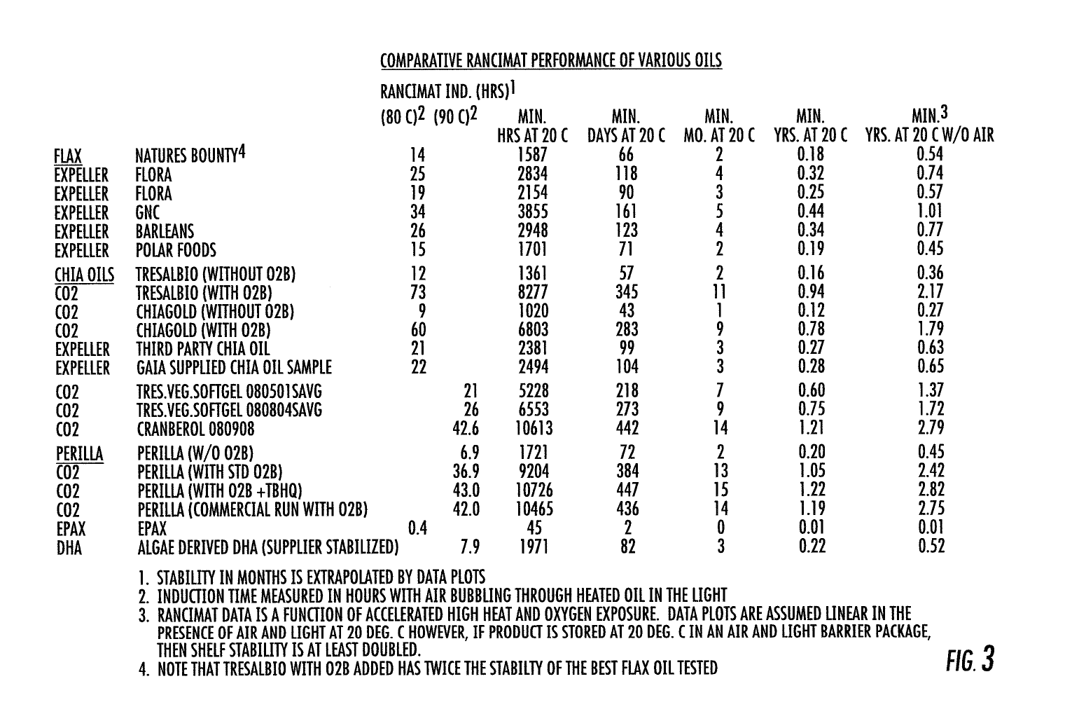

FIG. 3 is a chart showing a comparative Rancimat performance of various oils, including the chia seed oil extract such as produced as described above relative to the perilla seed oil extract as also produced as explained above. There is also illustrated flax seed oil and various values, including an algae oil.

There are noted differences between chia seed derived compositions and perilla seed compositions. These include not only the ratio of ALA:LA:oleic acid (Omega3:omega-6:omega-6), but also the differences between the post extracted (partially defatted) residues (ChiaMax and PerillaMax, respectively). ChiaMax produces a highly thixotropic goo in water. A spoon can stand up in hydrated ChiaMax, while PerillaMax has no thixotropic properties. Chia seed oil has about 32% CO2 extractive oil content while perilla seed has about 45% extractive oil content.

A substantial difference between the chia and perilla oils is the level of omega-9 (oleic acid, a mono-unsaturated acid), which is much more prevalent in perilla seed oil than in chia seed oil as the fatty acids profiles below show. Since perilla seed oil has a very high ratio of ALA to LA, it is more heart healthy in a Western diet. Such diets are typified by high levels of pro-inflammatory LA (omega-6) versus low levels of anti-inflammatory ALA ratios (omega-3). Also, because perilla seed oil extract has three times the amount of oleic acid as chia seed oil, it is even more heart healthy since oleic acid is known to lower LDL levels in man (Mediterranean diet). When LDL's are oxidized, this begins the atherosclerotic process.

The omega-3 ALA to omega-6 LA ratio is much higher in perilla seed oil than in chia seed oil and one reason is because the LA content in perilla seed has replaced a good portion of the LA that is evident in chia seed oils with oleic acid (omega-9).

When comparing Rancimat data between CO2 extracted chia seed oil and CO2 extracted perilla seed oil, despite a high level of unsaturated fatty acids present in extracted perilla seed, it is more stable than chia seed oil both with and without added anti-oxidants (O2B). Both oils contain the same relative percent of total unsaturated acids. However, perilla seed oil contains about 10% less polyunsaturated fatty acid content than chia seed oil. This helps explain why CO2 extracted perilla seed oil is intrinsically more stable than CO2 extracted chia seed oil as the attached Rancimat data in FIG. 3 shows.

Perilla seed oil (CO2 extracted):

| Component | % Average | % FA in Profile |
|---|---|---|
| Palmitic | 5.8 | 6.4 |
| Stearic | 1.8 | 2.0 |
| Oleic | 19.4 | 21.3 |
| Linoleic | 8.8 | 9.7 |
| Linolenic | 55.0 | 60.6 |
| Total Fatty Acids | 90.8 | 100.0 |

Chia Seed Oil (CO2 extracted):

| Test | Protocol | Specs/Limits | Result | | Analyst |
|---|---|---|---|---|---|
| Water Content | VQP-050 | ≦1.5 | 0.1 | | MS |
| (%) | | | | | |
| Peroxide Value | VQP-050 | ≦10.0 | 1.3 | | MS |
| Palmitic Acid | VQP-050 | n/a | 6.4 | 7.0 | GM |
| (%) | | | | | |
| Stearic Acid | VQP-050 | n/a | 3.6 | 3.9 | GM |
| (%) | | | | | |
| Oleic Acid (%) | VQP-050 | n/a | 7.6 | 8.3 | GM |
| Linoleic Acid | VQP-050 | n/a | 20.1 | 21.9 | GM |
| (%) | | | | | |
| Linolenic Acid | VQP-050 | n/a | 54.0 | 58.9 | GM |
| (%) | | | | | |
| Total Fatty | VQP-050 | 85.0-95.0 | 91.7 | 100.0 | GM |
| Acids (%) | | | | | |

The production diagram in FIG. 2 shows the process used to obtain the perilla seed extract in accordance with a non-limiting example. It should be understood that extraction technology creates materials for human nutrition and supplementation and offers various benefits including enhanced efficacy with the isolation of key components to allow higher dosage and targeted performance. Extraction allows standardization. Natural materials tend to vary in make-up and extraction makes them consistent. It is also convenient because smaller dosages of high efficiency materials allow a consumer to more easily obtain the required levels of nutrients in a daily regimen. There is also enhanced safety because extraction gives more of the desired products and less of what is not desired. Extraction allows the removal of compounds that are not optimal for human health from natural materials.

The desired extraction technology for the perilla seed oil extract and other oils from seeds as desired uses the DEEP EXTRACT® process from Valensa International as an ultra high pressure carbon dioxide extraction technology that yields micronutrients and has high extraction efficiency to deliver more of the higher molecular weight compounds that more closely track the natural source materials. The process is flexible and allows for possible fractionation of the product if desired and delivery of specific compounds out of the raw material.

This extraction process, such as the DEEP EXTRACT® process, offers a more gentle treatment of high value raw materials at temperature levels that are substantially below those used in other expeller press processes and some chemical solvent extraction processes in the absence of oxygen. This reduces the degradation of liable compounds, chemical change of a component and the oxidation potential. The supercritical CO² process offers virtual sterilization of the finished product and biomass, which are untouched by chemical solvents and stay “natural” as before extraction. Selective fractionation as described above is advantageous. Pressure is a major tool used to tailor the resulting fractionated products for chia seed oil or perilla seed oil for product quality and efficient manufacturing. Because the CO² extraction is an all-natural and organic process, the final product is devoid of impurities and residues and delivers through the supercritical CO² extraction the high molecular weight compounds such as sterols, carotenoids and long chain alcohols.

Supercritical CO² extraction is advantageous over other methods used for extracting botanicals, including tinctures (usually alcohol extraction; steam distillation; expeller pressing, sometimes referred to as “cold pressing,” and chemical solvent extraction). Chemical solvent extraction technology using strong solvents and supercritical CO² technology using high pressures typically offer the most comprehension extraction of a botanical. Supercritical CO² extraction conducted under very high pressure is advantageous. When carbon dioxide gas (CO²) is compressed above 73 bar at a temperature above 31 degrees C. (87.8 degrees Fahrenheit), it transforms into a dense gas as supercritical CO², which has an extremely high solvating capacity and a power to extract constituents of botanicals. Its solvating capacity is a function of its density and by changing its density with pressure, the manufacturer is able to select the quality, quantity and specific principles of the targeted extract. Supercritical CO² is biologically compatible and generally regarded as safe (GRAS) by the FDA. It is also non-flammable and environmentally sound.

Any defatted cake resulting from the supercritical CO² process is typically viable and can be marketed or used for further processing in a wide range of human/food applications. Sometimes the defatted cake is the primary product and the oil is secondary as described in commonly assigned U.S. Pat. No. 8,652,544, the disclosure which is hereby incorporated by reference in its entirety. That application describes a chia seed composition as a composition of matter formed from a stable, defatted whole grain flour derived from salvia hispanica L. whole ground seed using a suitable solvent such as using supercritical CO² fluid extraction. This chia seed extraction demonstrates that after processing of supercritical CO², the extract cake contains in some examples virtually no fat or oil and the resultant powder is approximately 50% protein and 50% carbohydrates that exist essentially as fiber. The perilla flour residue as left over from oil extraction is also viable and disclosed in commonly assigned U.S. patent application Ser. No. 13/910,577 filed Jun. 5, 2013, the disclosure which is hereby incorporated by reference in its entirety.

The supercritical CO² process offers a gentle treatment of high value raw materials at temperature levels substantially below those used in expeller press in some chemical solvent extraction operations in the absence of oxygen. This reduces the degradation of labile compounds, chemical changing components and the oxidation potential. The supercritical CO² process offers virtual sterilization of the finished product and biomass that are untouched by chemical solvents and stay “natural” as they were before extraction. It also allows the advantage of fractionating the extracted components selectively with pressure tailoring the resulting fractionated products for product quality and efficient manufacturing. This addresses pesticide/insecticide residues and handle concerns about microorganisms that are present in expeller press materials.

The perilla seed oil extract as obtained by the process as shown in FIG. 2 and described above is dewatered and decantated and antioxidants added such as the Valensa OTB® per oxidation blocker system as a stabilizer to ensure that the botanical extract reaches a consumer in an efficacious and safe form. Stabilization with the OTB® components increases shelf life and continued product quality and is advantageous over using preservatives to stabilize natural materials, which is often seen as a negative by consumers. The OTB® per oxidation blocker system used by Valensa is 100% natural, non-GMO, and protects sensitive oils and particularly the highly unsaturated oils derived from fish and botanicals from the manufacture to consumption. The OTB® per oxidation blocker is a synergistic proprietary formulation of powerful natural compounds including astaxanthin, phenolic antioxidants and natural tocopherols such as described above. This technology prevents destructive oxidative, photochemical and rancification reactions. It protects expensive and sensitive compounds such as carotenoids and polyunsaturated fatty acids and can boost the effectiveness of other antioxidants such as vitamin E because it chemically quenches stable vitamin E free radicals. The antioxidants have in-vivo activity to protect both products and people.

Perilla has a number of essential oils. These are extracted from the leaves of perilla. About 50% to about 60% of perillaldehyde is responsible for so much of the aroma and taste of perilla. There are other terpenes such as limonene, caryophyllene and farnesene. There are other chemotypes such as perilla keytone (PK), esholzia keytone (EK), perillene (PL), and various phenylpropanoids such as myristicin, dillapiole and elemicin. Citral is a type rich in rosefuran. Perilla oil typically is obtained by pressing the seeds of perilla that contain about 35% to about 45% oil. In some parts of Asia, perilla oil is an edible oil valued for medicinal benefit. Typically, perilla oil is a rich source of omega-3 fatty acid alpha-linolenic acids. As a drying oil, it is similar to tung oil or linseed oil and is sometimes used in paint, varnish, linoleum, printing ink, lacquers and other protective waterproof coatings. In Japan, the oxime of perillaldehyde (perillartin) is used as an artificial sweetener and typically is about 2,000 times sweeter than sucrose.

Rancimat testing has shown the advantages of the perilla seed oil extract in accordance with a non-limiting example. This method is an accelerated oxidation test that is a simple, quick and efficient way to screen the effectiveness of the antioxidants used in liquid fats and oils. Typically, the rancimat test is an accelerated oxidation test in which the oil or fat to be tested is run at an elevated temperature exposing the sample to air accelerating the oxidation process of the oil. Auto oxidation typically occurs in a few hours instead of the months or years and the metabolites are driven off into a measuring vessel that measures the change in conductivity in one example. This would indicate the point at which the formation of volatile carboxylic acids and oxidation has occurred.

It is also possible to disperse nano- and/or micro-particles of rice or sugar cane based policosanol for providing a heart healthy dietary supplement. Such dietary supplement composition additives are disclosed in commonly assigned U.S. Pat. No. 7,959,950, the disclosure which is hereby incorporated by reference in its entirety. This human or animal dietary supplement composition includes one or more long chain (C24-C36) primary alcohols (policosanols) dispersed in food-grade oils or fats where the average policosanol particle size is greater than 2 microns and less than 10 microns in one example, and in another example, less than 100 microns.

The desired seed oil extract, such as the perilla seed oil extract, is combined with the different carotenoids, and in an example, phospholipids for enhanced absorption to form a composition and associated method for its use to treat or support eye health and/or cardiovascular. Different carotenoids can be used, including 3, 3′S-astaxanthin derived from Haematococcus pluvialis in combination with other carotenoids such as lutein, trans-zeaxanthin, and meso-zeaxanthin for eye health. For heart and cardiovascular health, coenzyme Q10 may be used as explained in detail below.

With respect to humans and eye health, carotenoids have been found important with their antioxidant properties. About ten carotenoids are found in human serum. The major carotenoids in human serum are beta-carotene, alpha-carotene, cryptoxanthin, lycopene and lutein. Small amounts of zeaxanthin, phytofluene, and phytoene are found in human organs. However, of the ten carotenoids found in human serum, only two, trans- and/or meso-zeaxanthin and lutein, have been found in the human retina. Zeaxanthin is the predominant carotenoid in the central macula or foveal region and is concentrated in the cone cells in the center of the retina, i.e., the fovea. Lutein is predominantly located in the peripheral retina in the rod cells. Therefore, the eye preferentially assimilates zeaxanthin over lutein in the central macula which is a more effective singlet oxygen scavenger than lutein. It has been theorized that zeaxanthin and lutein are concentrated in the retina because of their ability to quench singlet oxygen and scavenge free radicals, and thereby limit or prevent photic damage to the retina.

Therefore, only two of the about ten carotenoids present in human serum are found in the retina. Beta-carotene and lycopene, the two most abundant carotenoids in human serum, either have not been detected or have been detected only in minor amounts in the retina. Beta-carotene is relatively inaccessible to the retina because beta-carotene is unable to cross the blood-retinal brain barrier of the retinal pigmented epithelium effectively. It also is known that another carotenoid, canthaxanthin, can cross the blood-retinal brain barrier and reach the retina. Canthaxanthin, like all carotenoids, is a pigment and can discolor the skin. Canthaxanthin provides a skin color that approximates a suntan, and accordingly has been used by humans to generate an artificial suntan. However, an undesirable side effect in individuals that ingested canthaxanthin at high doses for an extended time was the formation of crystalline canthaxanthin deposits in the inner layers of the retina. Therefore, the blood-retinal brain barrier of the retinal pigmented epithelium permits only particular carotenoids to enter the retina. The carotenoids other than zeaxanthin and lutein that do enter the retina cause adverse effects, such as the formation of crystalline deposits by canthaxanthin, which may take several years to dissolve. Canthaxanthin in the retina also caused a decreased adaptation to the dark.

Investigators have unsuccessfully sought additional antioxidants to further counteract the adverse affects of singlet oxygen and free radical species on in the eye. The investigators have studied the antioxidant capabilities of several compounds, including various carotenoids. Even though the carotenoids are strong antioxidants, investigators have failed to find particular carotenoids among the 600 naturally-occurring carotenoids that effectively quench singlet oxygen and scavenge for free radical species, that are capable of crossing the blood-retinal brain barrier, that do not exhibit the adverse affects of canthaxanthin after crossing the blood-retinal brain barrier, and that ameliorate eye disease or injury and/or retard the progression of a degenerative disease of the eye and are more potent anti-oxidants than either lutein or zeaxanthin.

It is known that singlet oxygen and free radical species are significant contributors to central nervous system, and particularly eye injury and disease. For example, it has reported that consumption of an antioxidant, such as ascorbic acid (Vitamin C), alpha-tocopherol (Vitamin E) or beta-carotene (which is converted in vivo to lutein), can decrease the prevalence of age-related macular degeneration.

Several carotenoids, including astaxanthin, are strong antioxidants compared to beta-carotene, ascorbic acid and other widely used antioxidants in vitro. It is also known that particular carotenoids selectively cross the blood-retinal brain barrier, and that certain carotenoids other than zeaxanthin and lutein that cross the blood-retinal brain barrier cause adverse affects.

Astaxanthin is a more effective antioxidant than carotenoids such as zeaxanthin, lutein, tunaxanthin, canthaxanthin, beta-carotene, and alpha-tocopherol in vitro. For example, some in vitro and in vivo studies with respect to astaxanthin demonstrated that the mean effective concentration of astaxanthin which inhibits lipid peroxidation was 500 times lower than that of alpha-tocopherol. In vitro, astaxanthin exhibits a strong quenching effect against singlet oxygen and a strong scavenging effect against free radical species. This free radical theory of retinal damage has been advanced by investigators examining the effectiveness of various antioxidants in ameliorating these diseases.

To date, investigative efforts have been directed to preventing diseases and injury because the resulting free radical-induced damage is not effectively treatable. Therefore, a need exists for a method not only to prevent or retard, but also to ameliorate, degenerative and traumatic diseases and injuries to the central nervous system, and particularly the eye. It has been found that a therapeutically effective amount of a multi-ingredient composition includes a seed oil extract, such as from perilla or other seed, and mixed carotenoids comprising at least astaxanthin and desirably S, S′-astaxanthin derived from Haematococcus pluvialis in an example, and one or more of lutein and/or trans-zeaxanthin or meso-zeaxanthin. Also, phospholipids may be added to enhance bioavailability of the carotenoids and used in combination with the seed oil extract or alone without the seed oil extract. The composition includes 50 to 1,000 mg of oil, and in a preferred example, 50 to 500 mg of oil, 0.5 to 8 mg of astaxanthin, 2 to 15 mg of lutein and 0.2 to 12 mg of trans-zeaxanthin. The oil may be derived from the perilla or other seed. The oil may include phospholipids as described below either whole or in part, including that derived from a roe extract. An example of the composition includes 4 mg of astaxanthin, about 10 mg of lutein, and 1.2 mg of trans-xeazanthin. This dietary supplement composition is formulated into a single dosage capsule in an example. In another example, the 3, 3′S-astaxanthin derived from Haematococcus pluvialis is 0.1 to 16% by weight of the seed oil extract and/or phospholipids, including roe extract, the lutein is 0.4 to 30% by weight of the seed oil extract and/or phospholipids, and the trans-zeaxanthin is 0.04 to 24% by weight of the seed oil extract and/or phospholipids. It is possible to supplement krill oil or an algae based oil as explained below to the seed oil extract and/or phospholipids. Krill oil will give greater phospholipids usually.

As noted before, the phospholipids could be formed from egg yolk or seed oil phospholipids and include many different plant based phospholipids that remove an omega-3 platform. The composition may include about 40-60% phospholipids having little phospholipid bound EPA or DHA. Sunflower, safflower, and soybean could be used as sources of the phospholipids. An example is the phospholipid rich egg roe extract, and it can be admixed with a seed oil and the composition may include at least 50% phospholipids. If it is combined with fish oil having EPA and DHA, the amount of DHA may be at least 50% greater than the amount of EPA. The composition may have about 30% triacylglycerides in an example. The phospholipids may be used alone with the carotenoids without any other type of seed oil extract, for example, phospholipids derived from a marine source or a plant base source.

The astaxanthin is preferably derived from Haematococcus pluvialis algae, Pfaffia, krill or by synthetic routes, in the free diol, monoester or diester form, for example, at a daily dose of about 0.5 to 8 mg and up to 12 mg, with varying ranges in between, and in one example, 2 to 4 mg.

It should be understood that the leading cause of visual loss among elderly persons is dry or atropic AMD, which has an increasingly important social and economic impact in the United States. As the size of the elderly population increases in this country, AMD will become a more prevalent cause of blindness than both diabetic retinopathy and glaucoma combined. Although laser treatment has been shown to reduce the risk of extensive macular scarring from the “wet” or neovascular form of the disease, there are currently no effective treatments for the vast majority of patients with wet AMD.

The Eye Diseases Prevalence Research Group (EDPRG) attributes AMD as the major cause of blindness among elderly people of European ancestry. Among white persons, AMD is believed to account for more than 50% of all blinding conditions.

The EDPRG estimates that approximately 1.2 million residents of the US are living with neovascular AMD and 970,000 are living with geographic atrophy, while 3.6 million are living with bilateral large drusen. In the next 20 years, these values are expected to increase by 50% with projected demographic shifts.

Age-related developmental changes in retinal morphology and energy metabolism, as well as cumulative effects of environmental exposures may render the neural and vascular retina and retinal pigment epithelium more susceptible to damage in late adulthood. Along with these metabolic and structural changes and exposures, the aging eye also experiences a reduction in the potency of endogenous and exogenous defense systems. Pharmacological and surgical treatment options are of limited scope and efficacy currently. They are costly and may result in complications as severe as end-stage disease. The likelihood of vision loss among persons with neovascular AMD can be reduced with anti-VEGF treatment, photodynamic therapy, and laser photocoagulation.

Nutrient-based preventative treatments for AMD development and progression have been examined in several studies including AREDSI, a NEI-sponsored study, the LAST, TOZAL and CARMIS studies for example. AREDS was a multi-center study of the natural history of AMD and cataract. AREDS included a controlled randomized clinical trial designed to evaluate the effect of pharmacological doses of zinc and/or a formulation containing nutrients with antioxidant properties (vitamin C, vitamin E, and β-carotene) on the rate of progression to advanced AMD and on visual acuity outcomes. The use of the combination of antioxidants and zinc reduced the risk of development of advanced AMD in participants who had at least a moderate risk of developing AMD by about 25%. The overall risk of moderate vision loss [≧15 letters on the Early Treatment Diabetic Retinopathy Study (ETDRS) chart] was reduced by 19% at 5 years.

Of approximately 600 carotenoids identified in nature in the human diet, and 20 in human serum, only two forms of dietary xanthophylls, lutein and zeaxanthin, are present in human macular pigment. Lutein represents approximately 36% of all retinal carotenoids; zeaxanthin and meso-zeaxanthin each represent about 18%.

The natural tissue distribution, biochemical, and biophysical characteristics of lutein provide a reasonable basis for speculating that this nutrient acts in biological systems as: (1) an important structural molecule within cell membranes; (2) a short-wavelength light filter; (3) a modulator of intra- and extracellular reduction-oxidation (redox) balance; and (4) a modulator in signal transduction pathways. Lutein and zeaxanthin were considered for inclusion in the AREDS formulation; however, at the time of AREDS' initiation, neither carotenoid was readily available for manufacturing in a research formulation.

The evidence base suggests that macular xanthophylls in combination with omega-3 LCPUFAs from fish oil may act as modifiable factors capable of modulating processes implicated in existing AMD pathogenesis and progression and is the basis for the on-going US Government sponsored AREDS II study. Intake of these compounds may also show merit as a well-tolerated preventive intervention. Biochemical and biophysical properties of these compounds demonstrate a capacity to modulate factors and processes that activate and are activated by exposures associated with aging. These exposures include developmental changes associated with aging, chronic light exposure, alterations in energy metabolism, and cellular signaling pathways.

### Dry Eye Syndrome

According to C Stephen Foster, M D, FACS, FACR, FAA0, Clinical Professor of Ophthalmology, Harvard Medical School; Consulting Staff, Department of Ophthalmology, Massachusetts Eye and Ear Infirmary; Founder and President, Ocular Immunology and Uveitis Foundation, Massachusetts Eye Research and Surgery Institution et al, dry eye is a very common disorder affecting a significant percentage (approximately 10-30%) of the population, especially those older than 40 years.

In the United States, an estimated 3.23 million women and 1.68 million men, a total of 4.91 million people, aged 50 years and older are affected.

Dry eye is a multi-factorial disease of the tears and the ocular surface that results in symptoms of discomfort, visual disturbance, and tear film instability with potential damage to the ocular surface. Dry eye is accompanied by increased osmolarity of the tear film and inflammation of the ocular surface.

The tear layer covers the normal ocular surface. Generally, it is accepted that the tear film is made up of 3 intertwined layers, as follows:

1) A superficial thin lipid layer (0.11 μm) is produced by the meibomian glands, and its principal function is to retard tear evaporation and to assist in uniform tear spreading.

2) A middle thick aqueous layer (7 μm) is produced by the main lacrimal glands (reflex tearing), as well as the accessory lacrimal glands of Krause and Wolfring (basic tearing).

3) An innermost hydrophilic mucin layer (0.02-0.05 μm) is produced by both the conjunctiva goblet cells and the ocular surface epithelium and associates itself with the ocular surface via its loose attachments to the glycocalyx of the microplicae of the epithelium. It is the hydrophilic quality of the mucin that allows the aqueous to spread over the corneal epithelium.

The lipid layer produced by the meibomian glands acts as a surfactant, as well as an aqueous barrier (retarding evaporation of the underlying aqueous layer), and provides a smooth optical surface. It may also act as a barrier against foreign particles and may also have some antimicrobial properties. The glands are holocrine in nature, and so the secretions contain both polar lipids (aqueous-lipid interface) and nonpolar lipids (air-tear interface) as well as proteinaceous material. All of these are held together by ionic bonds, hydrogen bonds, and van der Waals forces. The secretions are subject to neuronal (parasympathetic, sympathetic, and sensory sources), hormonal (androgen and estrogen receptors), and vascular regulation. Evaporative loss is predominantly due to meibomian gland dysfunction (MGD).

The aqueous component is produced by the lacrimal glands. This component includes about 60 different proteins, electrolytes, and water. Lysozyme is the most abundant (20-40% of total protein) and also the most alkaline protein present in tears. It is a glycolytic enzyme that is capable of breaking down bacterial cell walls. Lactoferrin has antibacterial and antioxidant functions, and the epidermal growth factor (EGF) plays a role in maintaining the normal ocular surface and in promoting corneal wound healing. Albumin, transferrin, immunoglobulin A (IgA), immunoglobulin M (IgM), and immunoglobulin G (IgG) are also present.

Aqueous tear deficiency (ATD) is the most common cause of dry eye, and it is due to insufficient tear production. The secretion of the lacrimal gland is controlled by a neural reflex arc, with afferent nerves (trigeminal sensory fibers) in the cornea and the conjunctiva passing to the pons (superior salivary nucleus), from which efferent fibers pass, in the nervus intermedius, to the pterygopalatine ganglion and postganglionic sympathetic and parasympathetic nerves terminating in the lacrimal glands.

Keratoconjunctivitis sicca (KCS) is the name given to this ocular surface disorder. KCS is subdivided into Sjogren syndrome (SS) associated KCS and non-SS associated KCS. Patients with aqueous tear deficiency have SS if they have associated xerostomia and/or connective tissue disease. Patients with primary SS have evidence of a systemic autoimmune disease as manifested by the presence of serum auto-antibodies and very severe aqueous tear deficiency and ocular surface disease. These patients, mostly women, do not have a separate, identifiable connective tissue disease. Subsets of patients with primary SS lack evidence of systemic immune dysfunction, but they have similar clinical ocular presentation. Secondary SS is defined as KCS associated with a diagnosable connective tissue disease, most commonly rheumatoid arthritis but also SLE and systemic sclerosis.

Non-SS KCS is mostly found in postmenopausal women, in women who are pregnant, in women who are taking oral contraceptives, or in women who are on hormone replacement therapy (especially estrogen only pills). The common denominator here is a decrease in androgens, either from reduced ovarian function in the postmenopausal female or from increased levels of the sex hormone binding globulin in pregnancy and birth control pill use. Androgens are believed to be trophic for the lacrimal and meibomian glands. They also exert potent anti-inflammatory activity through the production of transforming growth factor beta (TGF-beta), suppressing lymphocytic infiltration.

Lipocalins (previously known as tear-specific prealbumin), which are present in the mucous layer, are inducible lipid-binding proteins produced by the lacrimal glands that lower the surface tension of normal tears. This provides stability to the tear film and also explains the increase in surface tension that is seen in dry eye syndromes characterized by lacrimal gland deficiency. Lipocalin deficiency can lead to the precipitation in the tear film, forming the characteristic mucous strands seen in patients with dry eye symptomatology.

The glycocalyx of the corneal epithelium contains the transmembrane mucins (glycosylated glycoproteins present in the glycocalyx) MUC1, MUC4, and MUC16. These membrane mucins interact with soluble, secreted, gel-forming mucins produced by the goblet cells (MUC5AC) and also with others like MUC2. The lacrimal gland also secretes MUC7 into the tear film.

These soluble mucins move about freely in the tear film (a process facilitated by blinking and electrostatic repulsion from the negatively charged transmembrane mucins), functioning as clean-up proteins (picking up dirt, debris, and pathogens), holding fluids because of their hydrophilic nature, and harboring defense molecules produced by the lacrimal gland. Transmembrane mucins prevent pathogen adherence (and entrance) and provide a smooth lubricating surface, allowing lid epithelia to glide over corneal epithelia with minimal friction during blinking and other eye movements. Recently, it has been suggested that the mucins are mixed throughout the aqueous layer of tears (owing to their hydrophilic nature) and, being soluble, move freely within this layer.

Mucin deficiency (caused by damage to the goblet cells or the epithelial glycocalyx), as seen in Stevens-Johnson syndrome or after a chemical burn, leads to poor wetting of the corneal surface with subsequent desiccation and epithelial damage, even in the presence of adequate aqueous tear production.

### Pathophysiology

A genetic predisposition in SS associated KCS exists as evident by the high prevalence of human leukocyte antigen B8 (HLA-B8) haplotype in these patients. This condition leads to a chronic inflammatory state, with the production of auto-antibodies, including antinuclear antibody (ANA), rheumatoid factor, fodrin (a cytoskeletal protein), the muscarinic M3 receptor, or SS-specific antibodies (eg, anti-RO [SS-A], anti-LA [SS-B]), inflammatory cytokine release, and focal lymphocytic infiltration (i.e., mainly CD4⁺ T cells but also B cells) of the lacrimal and salivary gland, with glandular degeneration and induction of apoptosis in the conjunctiva and lacrimal glands. This results in dysfunction of the lacrimal gland, with reduced tear production, and loss of response to nerve stimulation and less reflex tearing. Active T lymphocytic infiltrate in the conjunctiva also has been reported in non-SS associated KCS.

Both androgen and estrogen receptors are located in the lacrimal and meibomian glands. SS is more common in postmenopausal women. At menopause, a decrease in circulating sex hormones (i.e., estrogen, androgen) occurs, possibly affecting the functional and secretory aspect of the lacrimal gland. Forty years ago, initial interest in this area centered on estrogen and/or progesterone deficiency to explain the link between KCS and menopause. However, recent research has focused on androgens, specifically testosterone, and/or metabolized androgens.

It has been shown that in meibomian gland dysfunction, a deficiency in androgens results in loss of the lipid layer, specifically triglycerides, cholesterol, monounsaturated essential fatty acids (e.g., oleic acid), and polar lipids (eg, phosphatidylethanolamine, sphingomyelin). The loss of polar lipids (present at the aqueous-tear interface) exacerbates the evaporative tear loss, and the decrease in unsaturated fatty acids raises the melting point of meibum, leading to thicker, more viscous secretions that obstruct ductules and cause stagnation of secretions. Patients on anti-androgenic therapy for prostate disease also have increased viscosity of meibum, decreased tear break-up time, and increased tear film debris, all indicative of a deficient or abnormal tear film.

It is known that in various tissues pro-inflammatory cytokines may cause cellular destruction. For example including interleukin 1 (IL-1), interleukin 6 (IL-6), interleukin 8 (IL-8), TGF-beta, TNF-alpha, and RANTES, are altered in patients with KCS. IL-1 beta and TNF-alpha, which are present in the tears of patients with KCS, cause the release of opioids that bind to opioid receptors on neural membranes and inhibit neurotransmitter release through NF-K β production. IL-2 also binds to the delta opioid receptor and inhibits cAMP production and neuronal function. This loss of neuronal function diminishes normal neuronal tone, leading to sensory isolation of the lacrimal gland and eventual atrophy.

Pro-inflammatory neurotransmitters, such as substance P and calcitonin gene related peptide (CGRP), are released, which recruit and activate local lymphocytes. Substance P also acts via the NF-AT and NF-K β signaling pathway leading to ICAM-1 and VCAM-1 expression, adhesions molecules that promote lymphocyte homing and chemotaxis to sites of inflammation. Cyclosporin A is an NK-1 and NK-2 receptor inhibitor that can down-regulate these signaling molecules and is a novel addition to the therapeutic armamentarium for dry eye, being used to treat both aqueous tear deficiency and meibomian gland dysfunction. It has been shown to improve the goblet cell counts and to reduce the numbers of inflammatory cells and cytokines in the conjunctiva.

These pro-inflammatory cytokines, in addition to inhibiting neural function, may also convert androgens into estrogens, resulting in meibomian gland dysfunction, as discussed above. An increased rate of apoptosis is also seen in conjunctival and lacrimal acinar cells, perhaps due to the cytokine cascade. Elevated levels of tissue-degrading enzymes called matrix metalloproteinases (MMPs) are also present in the epithelial cells.

Mucin synthesizing genes, designated MUC1-MUC17, representing both transmembrane and goblet-cell secreted, soluble mucins, have been isolated, and their role in hydration and stability of the tear film are being investigated in patients with dry eye syndrome. Particularly significant is MUC5AC, expressed by stratified squamous cells of the conjunctiva and whose product is the predominant component of the mucous layer of tears. A defect in this and other mucin genes may be a factor in dry eye syndrome development. In addition to dry eye, other conditions, such as ocular cicatricial pemphigoid, Stevens-Johnson syndrome, and vitamin A deficiency, which lead to drying or keratinization of the ocular epithelium, eventually lead to goblet cell loss. Both classes of mucins are decreased in these diseases, and, on a molecular level, mucin gene expression, translation, and posttranslational processing are altered.

Normal production of tear proteins, such as lysozyme, lactoferrin, lipocalin, and phospholipase A2, is decreased in KCS.

It is clear from the above discussion that common causes of dry eye syndromes may be ameliorated by treatment with anti-inflammatory agents such as topical corticosteroids, topical cyclosporine A and/or topical/systemic omega-3 fatty acids.

Studies that examined the association of dietary lutein/zeaxanthin intake with advanced AMD have yielded inverse relationships that are statistically significant. The magnitude of odds ratios in these studies ranged from 0.1 to 0.7. Both sets of findings are germane in guiding applied clinical research on prevention and treatment of retinal disease, since: (1) tissue concentrations of DHA, lutein, and zeaxanthin per unit area are substantially higher in the retina than elsewhere in the body; and (2) retinal tissue status of these compounds is modifiable and dependent upon intake.

The AREDS II study protocol (concluded its scientific rational by stating: “There is a compelling need to implement a clinical trial on nutrients that are both concentrated in the retina and implicated in modulation of pathogenic factors and processes of AMD.”

It has been well established that lutein and trans-zeaxanthin are present in human retinal tissue and that they function to protect the eye from photo induced injury. The CARMIS study, which included a mixture of lutein, trans-zeaxanthin and astaxanthin, is the only clinical trial which reported the use of astaxanthin. Unfortunately, there have been no reports of the use of astaxanthin alone in any human clinical trial for the prevention or amelioration of dry AMD. The CARMIS study failed to determine if supplementation with astaxanthin alone is a key determinate of the positive outcomes of the study or that astaxanthin deposited on retinal epithelial cells. One possible interpretation of the CARMIS study is that lutein and zeaxanthin alone provided the observed benefits of the formulation employed, or in another interpretation that astaxanthin in combination with lutein and zeaxanthin provided the observed benefits. However, in no possible interpretation can one conclude unequivocally that astaxanthin alone prevents or ameliorates dry AMD.

In addition, the work of Tso et al. as in U.S. Pat. No. 5,527,533, the disclosure which is hereby incorporated by reference in its entirety, and as disclosed in the article entitled, “Experiments on Visual Cells by Nature and Man: In Search of Treatment for Photoreceptor Degeneration,” Investigative Opthalmology and Visual Science, 30(12), pages 2421-2454 (December 1989), though claiming utility of astaxanthin for prevention or amelioration of dry AMD in humans, was not based on clinical trials performed on human subjects but instead on a different mammalian species, namely in rats.

Therefore, there remains no conclusive evidence that astaxanthin alone can prevent or ameliorate dry AMD in man since no human study has ever been performed using astaxanthin supplementation alone, nor has any human study shown that astaxanthin actually deposits anywhere in the human retina, the first required step to retinal protection by this powerful carotenoid.

### Potential Roles of Polyunsaturated Fatty Acids in Eye Physiology

An inverse relationship of dietary omega-3 LCPUFA intake with advanced AMD has been reported in six studies examining the issue. For prevalent disease, the magnitude of odds ratios for highest versus lowest omega-3 LCPUFA intake ranged from 0.4 to 0.9.

Among these studies, the one containing the largest number of subjects with neovascular or “wet” AMD yielded a significantly lower likelihood of having the disease among participants reporting the highest consumption of omega-3.

The scientific literature is replete with the certain human benefits of triacylglyceride bound EPA and DHA found in fish oil and fish oil concentrates and more recently the potential utility of phospholipid bound EPA and DHA found in krill oil derived from Euphasia superba or Antarctic krill.

The cardiovascular benefits as well as the anti-inflammatory benefits of such fish and krill oils, and in particular triacylglyceride bound EPA and DHA derived from fish oils as well as algae derived triacylglyceride bound DHA are well known. Such algae derived DHA is used in large part as a supplement in infant formulas to ensure brain health in the developing fetus and in infants. Seed oils are also a potent source as explained above.

LCPUFAs affect factors and processes implicated in the pathogenesis of vascular and neural retinal disease. Evidence characterizing structural and functional properties of LCPUFAs indicates that these nutrients may operate both as: (1) essential factors in the visual-sensory process, and (2) protective agents against retinal disease.

Docosahexaenoic Acid (DHA) is the major structural lipid of retinal photoreceptor outer segment membranes. Tissue DHA status affects retinal cell signaling mechanisms involved in phototransduction. Tissue DHA insufficiency is associated with conditions characterized by alterations in retinal function, and functional deficits have been ameliorated with DHA supplementation in some cases. Biophysical and biochemical properties of DHA may affect photoreceptor function by altering membrane permeability, fluidity, thickness, and lipid phase properties. DHA may operate in signaling cascades to enhance activation of membrane-bound retinal proteins. DHA may also be involved in rhodopsin regeneration.

DHA and Eicosapentaenoic Acid (EPA) may serve as protective agents because of their effect on gene expression, retinal cell differentiation, and survival. DHA activates a number of nuclear hormone receptors that operate as transcription factors for molecules that modulate redox-sensitive and proinflammatory genes; these include the peroxisome proliferator-activated receptor-α (PPAR-α) and the retinoid X receptor (RXR). In the case of PPAR-α, this action is thought to prevent endothelial cell dysfunction and vascular remodeling through inhibition of vascular smooth muscle cell proliferation, inducible nitric oxide synthase production, interleukin(IL)-1 induced cyclooxygenase (COX)-2 production, and thrombin-induced endothelin-1 production.

Research on model systems demonstrates that omega-3 LCPUFAs also have the capacity to affect production and activation of angiogenic growth factors, arachidonic acid-based proangiogenic eicosanoids, and matrix metalloproteinases involved in vascular remodeling.

EPA depresses vascular endothelial growth factor (VEGF)-specific tyrosine kinase receptor activation and expression. VEGF plays an essential role in induction of endothelial cell migration and proliferation, microvascular permeability, endothelial cell release of metalloproteinases and interstitial collagenases, and endothelial cell tube formation. The mechanism of VEGF receptor down-regulation is believed to occur at the tyrosine kinase nuclear factor-kappa B (NFkB) site because EPA treatment causes suppression of NFkB activation. NFkB is a nuclear transcription factor that up-regulates COX-2 expression, intracellular adhesion molecule (ICAM), thrombin, and nitric oxide synthase. All four factors are associated with vascular instability. COX-2 drives conversion of arachidonic acid to a number of angiogenic and pro-inflammatory eicosanoids.

Although the mechanistic benefits of dietary supplementation with EPA and DHA polyunstaruated fatty acids in triacylglyceride form are well known, it remains speculative that such triacylglyceride bound EPA and DHA can improve vision. Such hypothesis is now under exploration under the National Eye Institute's 5-year AREDS II study.

### Cataracts

A cataract is an opacity, or clouding, of the lens of the eye. The prevalence of cataracts increases dramatically with age. It typically occurs in the following way. The lens is an elliptical structure that sits behind the pupil and is normally transparent. The function of the lens is to focus light rays into images on the retina (the light-sensitive tissue at the back of the eye).

In young people, the lens is elastic and changes shape easily, allowing the eyes to focus clearly on both near and distant objects. As people reach their mid-40s, biochemical changes occur in the proteins within the lens, causing them to harden and lose elasticity. This causes a number of vision problems. For example, loss of elasticity causes presbyopia, or far-sightedness, requiring reading glasses in almost everyone as they age.

In some people, the proteins in the lens, notably those called alpha crystallins, may also clump together, forming cloudy (opaque) areas called cataracts. They usually develop slowly over several years and are related to aging. In some cases, depending on the cause of the cataracts, loss of vision progresses rapidly. Depending on how dense they are and where they are located, cataracts can block the passage of light through the lens and interfere with the formation of images on the retina, causing vision to become cloudy.

Nuclear cataracts form in the nucleus (the inner core) of the lens. This is the most common variety of cataract associated with the aging process. Cortical cataracts form in the cortex (the outer section of the lens). Posterior subcapsular cataracts form toward the back of a cellophane-like capsule that surrounds the lens. They are more frequent in people with diabetes, who are overweight, or those taking steroids. Although the causes of cataract formation remain largely unknown, researchers have been focusing on particles called oxygen-free radicals as a major factor in the development of cataracts. They cause harm in the following way:

Oxygen-free radicals (also called oxidants) are molecules produced by natural chemical processes in the body. Toxins, smoking, ultraviolet radiation, infections, and many other factors can create reactions that produce excessive amounts of these oxygen-free radicals. When oxidants are overproduced, these chemical reactions can be very harmful to nearly any type of cell in the body. At times these reactions can even affect genetic material in cells.

Cataract formation is one of many destructive changes that can occur with overproduction of oxidants, possibly in concert with deficiencies of an important protective antioxidant called glutathione. Glutathione occurs in high levels in the eye and helps clean up these free radicals. One theory is that in the aging eye, barriers develop that prevent glutathione and other protective antioxidants from reaching the nucleus in the lens, thus making it vulnerable to oxidation. Sunlight consists of ultraviolet (referred to as UVA or UVB) radiation, which penetrates the layers of the skin. Both UVA and UVB have destructive properties that can promote cataracts. The eyes are protected from the sun by eyelids and the structure of the face (overhanging brows, prominent cheekbones, and the nose). Long-term exposure to sunlight, however, can overcome these defenses.

UVB radiation produces the shorter wavelength, and primarily affects the outer skin layers. It is the primary cause of sunburn. It is also the UV radiation primarily responsible for cataracts. Long-term exposure to even low levels of UVB radiation can eventually cause changes in the lens, including pigment changes, which contribute to cataract development. (UVB also appears to play a role in macular degeneration, an age-related disorder of the retina.) UVA radiation is composed of longer wavelengths. They penetrate more deeply and efficiently into the inner skin layers and are responsible for tanning. The main damaging effect of UVA appears to be the promotion of the release of oxidants. Cataracts are common side effects of total body radiation treatments, which are administered for certain cancers. This observation indicates that ionizing radiation, which produces large numbers of free radicals dramatically accelerates cataract formation.

Glaucoma and its treatments, including certain drugs (notably miotics) and filtering surgery, pose a high risk for cataracts. The glaucoma drugs posing a particular risk for cataracts including demecarium (Humorsol), isoflurophate (Floropryl), and echothiophate (Phospholine). Uveitis is chronic inflammation in the eye, which is often caused by an autoimmune disease or response. Often the cause is unknown. It is a rare condition that carries a high risk for cataracts. It is not clear whether nutrition plays a significant role in cataract development. Dark colored (green, red, purple, and yellow) fruits and vegetables usually have high levels of important plant chemicals (phytochemicals) and may be associated with a lower risk for cataracts.

In analyzing nutrients, researchers have focused on antioxidants and carotenids. Studies have not demonstrated that antioxidant vitamin supplements (such as vitamins C and E) help prevent cataracts. Lutein and zeaxanthin are the two carotenids that have been most studied for cataract prevention. They are xanthophylis compounds, which are a particular type of carotenid. Lutein and zeaxanthin are found in the lenses of the eyes. Some evidence indicates that xanthophyll-rich foods (such as dark green leafy vegetables) may help retard the aging process in the eye and protect against cataracts. However, there is not enough evidence to suggest that taking supplements with these carotenoids lowers the risk of cataract formation. Since little is known about the exact mechanism for formation of cataracts, it is not surprising that there are no known drugs or dietary supplements including the carotenoids that prevent cataract formation there remains a need to find a suitable preventative treatment to prevent or ameliorate further cataract formation. Since no drugs can reverse nor prevent cataract formation, the only current treatment suitable for advanced cataract in humans is lens replacement surgery.

The ability of a carotenoid to pass the blood-retinal brain barrier is important because carotenoids are not synthesized by the human body. The only source of carotenoids for humans is dietary intake. Furthermore, humans have a very limited ability to modify carotenoids. Therefore, the carotenoids accumulate in various organs in the ingested form. Accordingly, if a particular carotenoid is unable to cross the blood-retinal brain barrier, the carotenoid cannot accumulate in the retina and serve as an antioxidant.

Furthermore, some carotenoids that are not normal constituents of human plasma, but have the ability to cross the blood-retinal brain barrier, have demonstrated adverse effects on the retina. Canthaxanthin which is intentionally ingested to provide an artificial suntan has accumulated in the retina in the form of crystals and has temporarily affected eye adaptation to the dark. In addition, beta-carotene has a very limited ability to cross the blood-retinal brain barrier.

Therefore, even though the carotenoids are known as strong antioxidants and are present in abundant supply, the carotenoids have not been used for the treatment of central nervous system damage, or eye damage, caused by disease or injury. The carotenoids investigated to date either could not effectively cross the blood-retinal barrier (i.e., beta-carotene) or adversely affected the eye (i.e., canthaxanthin).

In accordance with a non-limiting example, the composition comprises a therapeutically effective amount of a multi-ingredient composition of mixed carotenoids including at least S, S′-astaxanthin derived from Haematococcus pluvialis, and one or more of lutein and/or trans-zeaxanthin or meso-zeaxanthin admixed with a therapeutically effective amount of seed oil extract such as from the perilla and/or a phospholipid such as desired from roe extract or other sources as will be described. In an example, the composition includes 50 to 500 mg of seed oil extract and/or phospholipid, 0.5 to 8 mg of astaxanthin, 2 to 15 mg of lutein and 0.2 to 12 mg of trans-zeaxanthin. The astaxanthin could be 0.5 to 12 mg in an example. With the use of the phospholipids, the absorption of the carotenoids is enhanced and a lower range can be used. It is possible to use the phospholipid alone with lutein to increase the absorption of lutein. It is also possible to use the phospholipid alone with coenzyme Q10 for cardiovascular and other benefits. Of course, the phospholipids can be mixed with the seed oil. The composition contains naturally-occurring compounds and is a potent antioxidant and anti-inflammatory composition, which can be is used in a method to ameliorate and retard, or prevent, cell damage in an individual suffering from a degenerative, inflammatory disease or injury to the eye. In accordance with another non-limiting example, the administration of a therapeutically-effective amount of the composition to an individual prevents, retards and/or ameliorates free radical-induced damage resulting from eye disease or injury. For example, damage to a retina can result from either photic injury, neurodegenerative disease or an ischemic insult followed by reperfusion. With respect to damage from photic injury, the composition decreases the loss of photoreceptor cells. With respect to damage from ischemic insult, the composition ameliorates the loss of ganglion cells and the inner layers of the retinal neuronal network.

None of the carotenes tested to date, and most of the xanthophylls tested to date do not pass through the blood brain barrier with a few notable exceptions. These exceptions include lutein, trans-zeaxanthin, canthaxanthin and astaxanthin.

Human serum typically contains about ten carotenoids. The major carotenoids in human serum include beta-carotene, alpha-carotene, cryptoxanthin, lycopene and lutein. Small amounts of zeaxanthin, phytofluene and phytoene are also found in human organs. However, of all of these carotenoids, only zeaxanthin and lutein are found in the human retina. In addition to certain carotenoids, the retina also has the highest concentration of polyunsaturated fatty acids of any tissue in the human body. These polyunsaturated fatty acids are highly susceptible to free radial and singlet oxygen induced decomposition. Therefore, there is a need to protect these polyunsaturated fatty acids, which make up a portion of the cellular membrane bi-layer, from photo induced free radical or singlet oxygen degradation.

It has been theorized that zeaxanthin and lutein are concentrated in the retina because of their ability to quench singlet oxygen and to scavenge free radicals, because they pass the blood and eye brain barriers and are required in the oxygen rich environment of the retina to prevent light mediated free radical damage to the retina.

In fact, zeaxanthin is the predominant carotenoid found in the central portion of the retina and more specifically is located in concentration in the retinal cones located in the central area of the retina (i.e., the macula). Lutein, on the other hand, is located in the peripheral area of the retina in the rod cells. Therefore, the eye preferentially accumulates zeaxanthin over lutein in the critical central macular retinal area, (zeaxanthin interestingly, is a much more effective singlet oxygen scavenger than lutein), where the greatest level of light impinges.

Biochemists have determined the exact, yet complicated, mechanism for light sensory response in the eye. It involves a key protein called rhodopsin whose structure includes a bound polyunsaturated compound called retinal (retinal is structurally related to vitamin A). When light enters the eye, cis-retinal isomerizes to all its all-trans isomer, causing disassociation of itself from its protein carrier. The disassociation triggers a complicated cascade leading to nerve based transmission of electrons to the brain via the optic nerve. All of this “photochemistry” takes a mere 200 femtoseconds to occur making it one of the fastest biochemical to electron transformations known.

Chemists have learned that the retina is highly susceptible to polymerization by localized free radicals and highly reactive singlet oxygen. Because the retina is a strong absorber of light and because the retina is highly vascularized and thus rich in dissolved oxygen, nature has provided zeaxanthin as the key retinal carotenoid for protection of the central foveal region of the retina from light induced damage at that point in the center of the retina where the most significant light impingement occurs.

Clinical studies in man indicate that photic injury is a cause of age related macular degeneration because of the cumulative effect of repeated photic insult leading to the gradual loss of photoreceptor cells.

There have been many clinical trials designed to support the supplementation of the diet with lutein, however, as of 2007, there appears to be no unequivocal evidence that lutein supplementation is necessary in eye healthcare despite its wide acceptance as a supplement. This may simply imply that supplementation with extra lutein is not necessary since it is a readily available xanthophyll in many vegetables. However, use of lutein with a phospholipid to enhance absorption has been beneficial. More recently, trans-zeaxanthin and meso-zeaxanthin have also entered the marketplace as an eye healthcare supplements which indeed makes sense. However, is there yet a better carotenoid meeting all the requirements associated with eye/blood/brain barrier transport, accumulation in the macula and capable of long term use? The answer is found in the xanthophyll astaxanthin.

Dr. Mark Tso, at the University of Illinois, has demonstrated that astaxanthin is one such naturally occurring antioxidant meeting all of these critical criteria in rats. Astaxanthin is the carotenoid xanthophyll responsible for the red color in salmon, lobster, krill, crab, other shell fish and in the micro algae Haematoccous pluvialis. The latter source has made astaxanthin readily available worldwide for such uses. U.S. Pat. No. 5,527,533 was issued to the Univ. of Ill. describing the use of astaxanthin more fully in eye related diseases and which is hereby incorporated by in its entirety.

In addition, astaxanthin is a much more powerful antioxidant than canthoaxanthin, beta-carotene, zeaxanthin, lutein and alpha-tocopherol. Some researchers have discovered that astaxanthin is 550 times more potent than alpha-tocopherol, 27.5 times more potent than lutein and 11 times more potent that beta-carotene in quenching singlet oxygen. In addition, other researchers discovered that natural astaxanthin is 14 times more potent than alpha-tocopherol, 54 times more potent that beta-carotene and 65 times more potent that ascorbic acid (Vitamin C) in scavenging oxygen free radicals. Thus, though there are dramatic differences in the potency of astaxanthin when comparing the quenching of singlet oxygen and the scavenging of oxygen free radicals, it is clear that astaxanthin compares very favorably to zeaxanthin and lutein, the two carotenoids that are found naturally in the retina.

There is one more aspect of carotenoids, namely that some carotenoids can act as pro-oxidants. This is important since a carotenoid with pro-oxidant capability actually causes oxidation to occur in the body when high concentrations are present in tissue. Another researcher showed that beta-carotene, lycopene and zeaxanthin can become pro-oxidants under certain conditions. Since humans already have an abundant source of lutein and trans-zeaxanthin in their diets from many vegetable sources and are already present in the human eye, it appears that astaxanthin with its unique qualifying properties, unlike lutein or trans-zeaxanthin, may be the eye healthcare supplement of choice. With astaxanthin's extraordinarily potent antioxidant properties, its ability to cross the blood brain/eye barrier and concentrate in the retinal macula in other mammalian species, without the side effects seen with canthaxanthin, and in light of Tso's contributions, astaxanthin, in a convenient dietary supplement presentation, may emerge as the pre-eminent new ingredient addition to lutein and/or zeaxanthin eye healthcare supplementation for the management of eye related oxidative stress and thus the prevention and mitigation of degenerative diseases of the eye such as age related macular degeneration (ARMD) and cataract formation if astaxanthin deposition can be experimentally confirmed in human retinal tissue.

In addition, Tso found that light induced damage, photo-receptor cell damage, ganglion cell damage and damage to neurons of the inner retinal layers can be prevented or ameliorated by the use of astaxanthin including neuronal damage from ischemic, photic, inflammatory and degenerative insult in rats. Tso discloses the use of astaxanthin across a wide range of eye diseases including age related macular degeneration, diabetic neuropathy, cystoid macular edema, central retinal arterial and veneous occlusion, glaucoma and inflammatory eye diseases such as retinitis, uveitis, iritis, keratitis and scleritis, all disease states common to eye insult by oxidative species such as free radicals however this work was never confirmed in humans.

Oral administration of astaxanthin confirms that it is at least transported into human blood stream, however, its deposition in human retinal tissue has never been confirmed.

Astaxanthin is the major pigment of certain micro algae and crustaceans. Astaxanthin is a lipid-soluble pigment primarily used for pigmenting cultured fish, like salmon, which must ingest astaxanthin to yield consumer-acceptable pink-colored salmon muscle. Astaxanthin also is an antioxidant which is about 100 to about 1000 times more effective than alpha-tocopherol.

The prime source of commercial S,S′-astaxanthin is micro algae, and, to a very small extent, is found in krill oil derived from Euphasia superba (Antarctic Krill). Astaxanthin also is available synthetically, however synthetic astaxanthin may not be safe for use in humans since it contains 3 known enantiomers including R,R′, R, S′ and S,S′ which are not easily nor economically separated two of which have unknown human safety data. The preferred naturally-occurring S,S′-astaxanthin in one example can be used in the composition and method of the present invention.

As previously noted, the retinal pigment epithelium protects the retina by providing a blood-retinal brain barrier. The barrier excludes plasma constituents that are potentially harmful to the retina. As also previously noted, the blood-retinal brain barrier permits lutein and zeaxanthin to enter the retina, and excludes other carotenoids present in human serum, including beta-carotene which is the most abundant carotenoid in human serum. Astaxanthin is not a naturally-occurring constituent in the retina. Therefore, the presence of a physiologically significant amount of astaxanthin in the retina of rats may illustrate the ability of astaxanthin to readily cross the blood-retinal brain barrier into the retina of humans. The optimal dose of the composition can be determined by a person skilled in the art after considering factors such as the disease or injury to be treated, the severity of the central nervous system damage by oral administration. The daily dose of composition can be administered daily or in accordance with a regimen determined by a person skilled in the art, with the length of treatment depending upon the severity and nature of the injury to the central nervous system, the need to improve accommodation or to control dry eye syndrome.

The composition can be administered to an individual orally such as in a single dose capsule. When administered orally, the composition, for example, can be in the form of a liquid preparation. The administration of the composition to an individual suffering from an eye injury or disease, such as free radical-induced injury, benefits the vision of the individual by preventing further photoreceptor cells from damage or destruction. The free radical-induced damage can be attributed to light-induced injury or to injury resulting from an ischemic insult and subsequent reperfusion or neurodegenerative diseases. The administration of astaxanthin also helps prevent and retard photic injury in addition to ameliorating photic injury.

The administration of the composition ameliorates photoreceptor cell damage that is light induced, and ameliorates ganglion cell damage that is induced by ischemic insult and subsequent reperfusion. The administration of astaxanthin also retards the progress of degenerative eye diseases and benefits the vision of individuals suffering from a degenerative eye disease, such as age-related macular degeneration.

The administration of the composition also provides a method of treating ischemic retinal diseases, such as diabetic retinopathy, cystoid macular edema, central retinal arterial occlusion, central retinal venous occlusion and glaucoma. In addition, the composition is useful in treating inflammatory diseases of the eye such as retinitis, uveitis, iritis, keratitis and scleritis wherein free radicals are produced in abundance, the prevention of cataracts and the treatment of certain causes of dry eye syndromes.

Therefore, the antioxidant properties of the composition, coupled with the ability of the composition to cross the blood-retinal brain barrier, admixed with anti-inflammatory sources of EPA and DHA and the lack of toxicity of the composition and the lack of adverse side effects associated with the composition, make the composition a useful composition to prevent or ameliorate such eye related diseases, dry eye syndrome and/or cataracts and dry eye syndromes.

The carotenoids admixed with the seed oil extract such as perilla or other seed sources and/or phospholipids in this example should be able to address not only the eye diseases known to be ameliorated by carotenoids but also the inflammatory diseases of the eye (including but not limited to dry eye syndrome) associated with the known anti-inflammatory activity of omega-3's.

Astaxanthin is also known to reduce C-Reactive Protein (C-RP) blood levels in vivo. For example, in human subjects with high risk levels of C-RP three months of astaxanthin treatment resulted in 43% drop in the patient population's serum C-RP levels a drop which is below the unacceptable cardiovascular event risk level. Astaxanthin is so powerful that it has been shown to negate the pro-oxidant activity of Vioxx in vitro, a COX-2 inhibitor belonging to the NSAIDS drug class which is known to cause cellular membrane lipid per-oxidation leading to heart attacks and strokes. For this reason Vioxx was subsequently removed from the US market by the FDA. Astaxanthin is also absorbed in vitro by lens epithelial cells where it suppresses UVB induced lipid per-oxidative mediated cell damage at umol/L concentrations. Reduction of C-Reactive protein (CRP), reduction of LDL oxidation and an increase in the omega-3 index in vivo would presumably all be important positive contributors to cardiovascular health since each are well know biomarkers for cardiovascular health risk.

Astaxanthin has an excellent safety record. A conducted study obtained the results as follows:
Oral LD 50: 600 mg/kg (rats);
NOAEL: 465 mg/kg (rats); or
Serum Pharmacokinetics: Stewart et al. 2008
1) T_{1/2}: 16 hours;
2) Tₘₐₓ: 8 hours;
3) Cₘₐₓ: 65 μg/L.

At eight weeks of supplementation at 6 mg per day, there was no negative effect in healthy adults. Astaxanthin has three prime sources, e.g., 3 mg astaxanthin per 240 g serving of non-farmed raised salmon or a 1% to 12% astaxanthin oleoresin or 1.5-2.5% beadlet derived from microalgae. Literature references pertinent to the above discussion can be found in Lee et al., Molecules and Cells 16(1): 97-105, 2003; Ohgami et al., Investigative Ophthalmology and Visual Science 44(6): 2694-2701, 2003; Spiller et al., J. of the American College of Nutrition 21(5): October 2002; and Fry et al., University of Memphis, Human Performance Laboratories, 2001 and 2004, Reports 1 and 2.

It should be understood that different proportions of ingredients and percentages in compositions can be used depending on end use applications and other environmental and physiological factors when treating a patient condition.

The astaxanthin may be derived from Haematococcus pluvialis algae, Pfaffia, krill, or by synthetic routes, in the known free diol, monoester or diester form, and in one example, at a daily dose of 0.5-8 mg, and in another example, 0.5 to 12 mg. When the astaxanthin is combined with the phospholipid, a reduced amount of astaxanthin may be used, such as from 0.5 to 4 mg, and in one example, 2.0 to 4.0 mg. Other ranges may be available depending on end use applications. The same applies with use of other carotenoids such as lutein when admixed with the phospholipid. The composition may also include an n-3 (omega-3) fatty acid rich oil derived from fish oil, algae oil, flax seed oil, or chia seed oil when the n-3 fatty acid comprises alpha-linolenic, stearidonic, eicosapentaenoic or docosapentaenoic acid, and in one example, a seed oil extract such as perilla is preferred and/or other phospholipids. The composition may include naturally-derived and synthetic antioxidants that are added to retard degradation of fatty acids and astaxanthin.

Although a perilla seed oil extract is disclosed in combination with the carotenoids, including astaxanthin, it should be understood that other oils may be used either singularly or in combination with the perilla seed oil extract and with the carotenoids as disclosed, for example, additional phospholipids, and in other examples, different marine oils including a fish oil and/or krill oil and a fish oil derived choline phospholipid bound EPA and DHA. The different oils may include a shrimp mince and/or shell derived oils. There could also be an algae based oil added. The phospholipids are, in one example, preferred to enhance bioavailability.

The oils from seed oil extract such as perilla may be supplemented with phospholipids. The composition may include EPA and DHA functionalized as marine phospholipids and acyltriglycerides derived from krill. Some of these components are explained in the following chart:

| Components | Percentage (%) |
|---|---|
| PHOSPHOLIPIDS | |
| PC, PE, PI, PS, SM, CL | >40 |
| OMEGA-3 (functionalized on PL) | >30 |
| Eicosapentaenoid Acid (EPA)* | >17 (15% in one example and |
| | 10% in another) |
| Docosahexaenoid Acid (DHA)+ | >11 (9% in one example and |
| | 5% in another) |

Phospholipids are supplemented with the perilla seed oil extract and carotenoid composition and/or phospholipids and may be produced from seed, egg yolk, plants, and Antarctic krill (euphausia superba), which is a zooplankton (base of food chain). It is one of the most abundant marine biomass of about 500 million tons according to some estimates. Antarctic krill breeds in the pure uncontaminated deep sea waters. It is a non-exploited marine biomass and the catch per year is less than or equal to about 0.02% according to some estimates.

The krill oil that could be supplemented, in one example, is derived from Euphasia spp., comprising Eicosapentaenoic (EPA) and Docosahexaenoic (DHA) fatty acids in the form of triacylglycerides and phospholipids, although not less than 1% EPA and 5% DHA has been found advantageous. In another example, the krill oil includes at least 15% EPA and 9% DHA, of which not less than 45% are in the form of phospholipids, and in one example, greater than 50%. The composition can be delivered advantageously for therapeutic results with 1-4,000 mg of krill oil delivered per daily dose.

The seed oil, such as from perilla, may also include an n-3 (omega-3) fatty acid rich oil derived from fish oil, algae oil, flax seed oil, or chia seed oil when the n-3 fatty acid comprises alpha-linolenic, stearidonic, eicosapentaenoic or docosapentaenoic acid. The composition may include naturally-derived and synthetic antioxidants that are added to retard degradation of fatty acids.

Details of a type of CO2 extraction and processing technology (as supercritical CO2 extraction) and peroxidation blocker technology that can be used are disclosed in commonly assigned U.S. Pat. Nos. 8,652,544 and 8,586,104, the disclosures which are hereby incorporated by reference in their entirety.

It is possible to add as a supplemental oil a fish oil derived, choline based, phospholipid bound omega-3 fatty acid mixture including phospholipid bound polyunsaturated EPA and DHA to improve blood lipid profiles and reduce LDL either alone or admixed with other ingredients, for example, an LDL peroxidation blocker. One commercially available example of a mixture of fish oil derived, choline based, phospholipid bound fatty acid mixture including polyunsaturated EPA and DHA is a phospholipid, omega-3 preparation, which is derived from natural fish oil. One example of such composition is described below:

Ingredients (g/100 g):

| Pure Marine Phospholipids | n.l.t. 15 |
|---|---|
| DHA* | n.l.t. 12 |
| EPA** | n.l.t. 7 |
| Omega-3 | n.l.t. 22 |
| Omega-6 | <3 |

*Docosahexaenoic acid
**Eicosapenteanoic acid

### Analytical Data:

| | |
|---|---|
| Peroxide value (meq/Kg) | n.m.t. 5 |
| Loss on Drying (g/100 g) | n.m.t. 2 |

### Physical Properties:

| | |
|---|---|
| Consistency | Viscous Liquid |

The composition may also be supplemented with an n-3 (omega-3) fatty acid rich oil derived from fish oil, algae oil, flax seed oil, or chia seed oil wherein the n-3 fatty acid source comprises alpha-linolenic, stearidonic, eicosapentaenoic or docosapentaenoic acid. The composition may include naturally-derived and synthetic antioxidants that are added to retard degradation of fatty acids such as tocopherols, tocotrienols, carnosic acid or Carnosol and/or astaxanthin.

It is now known that phospholipids as briefly described above will enhance the absorption of certain carotenoids, including astaxanthin and lutein. It may also be used to enhance the absorption of coenzyme Q10. For example, one study as a single-dose, randomized and controlled trial indicated that the plasma lutein levels as measured at baseline increased incrementally up to 24 hours after ingestion of a krill oil relative to a comparable product that had relatively no phospholipids. The study indicated that the maximal lutein plasma absorption is higher in the krill oil and phospholipid ingestion than the comparator subjects that showed little change in lutein absorption. The comparator product contained equal amounts of lutein esters such as 6 mg, but did not include the krill or phospholipids to enhance absorption of any lutein as in the krill/phospholipids with the lutein. Thus, the phospholipids and possible antioxidant content probably aided to increase bioavailability of the ingredients, including lutein. It has also been determined that the phospholipid molecular structure will allow the body to better absorb krill oil and any DHA and EPA omega-3 fatty acids up to 2.5 times in certain instances compared to the triglyceride form most common in fish oils. This may aid absorption of other ingredients as well. Lutein is especially in the form of lutein esters and the current composition may also include vitamin A and thiamine such as vitamin B1 such that the enhancement with the perilla seed oil and/or phospholipids will be beneficial.

Lutein is a xanthophyll and found in green leafy vegetables such as spinach, kale and yellow carrots and modulates light energy and acts as non-photochemical quenching agents. It may be derived from egg yolks and animal fats in some examples. It is known that the human retina accumulates lutein and zeaxanthin. Zeaxanthin predominates the macula lutea while lutein may predominate elsewhere in the retina and serve as a photo-protectant for the retina from the damaging effects of free radicals produced by blue light, especially. Lutein is isomeric with zeaxanthin by differing in the placement of one double bond. It is a lipophilic molecule and generally insoluble in water with its long chromophore of conjugated double bonds as polyene chain. It has distinctive light-absorbing properties and works well with phospholipids, and thus, the added phospholipids as described below will operate to enhance absorption, acting with the perilla seed oil extract if used. In plants, it is present as fatty acid esters with one or two fatty acids bound to two hydroxyl groups and saponification as the de-esterification of lutein esters will yield free lutein from 1:1 to 1:2 molar ratio.

The AREDS2 study as a five-year test indicated that the omega-3 fatty acids may not be as beneficial as desired. It has not been determined that phospholipids can enhance absorption multiple times and up to eight times, according to some studies. The same may occur with zeaxanthin. It should be understood that lutein and zeaxanthin have identical chemical formulas and are isomers, but not stereoisomers and a major difference is the location of the double bond in one of the end rings. Lutein has three chiral centers and zeaxanthin has two chiral centers. The 3R, 3′S and 3S, 3′R stereoisomers of zeaxanthin are identical and therefore zeaxanthin has only three stereoisomeric forms. The 3R, 3′S stereoisomer is called meso-zeaxanthin. The natural form of zeaxanthin is 3R, 3′R zeaxanthin and the macula mainly contains the 3R, 3′R and meso-zeaxanthin forms, but smaller amounts of the 3S, 3′S form. Some proteins will circulate zeaxanthin and lutein for a better uptake within the macula and phospholipids operate as noted before.

It is possible to use natural or synthetic zeaxanthin such as prepared by a Wittig reaction that yields 96-98% of trans-(3R, 3R)-zeaxanthin and minor quantities of cis-zeaxanthin. Zeaxanthin is insoluble in water generally and somewhat soluble in ethanol like other carotenoids and soluble in chloroform. The hydroxyl groups on two of the outermost carbon atoms would make xanthophylls such as zeaxanthin more water soluble than other very hydrophobic carotenoids.

It is also possible to use the phospholipid with or without the seed oil extract to enhance absorption of coenzyme Q10, i.e., ubiquinone also referred to as COQ10 or Q10. The quinone chemical group refers to the 1, 4-benzoquinone as part of the quinone chemical group, while 10 is the isoprenyl chemical subunits. As is known to those skilled in the art, it is an oil-soluble component of the electron transport chain and is active in the aerobic cellular respiration and generates energy in the form of ATP. It has 3 redox states as fully oxidized, semi-quinone and full reduced. For that reason, it is excellent in the functions for the electron transport chain.

A dosage may be about 500 to about 1,200 mg/day and 100 to 500 mg/day depending on phospholipid absorption. Another range is 50 to 500 mg/day or 10 to 100 mg/day. Phospholipids would help reduce the amounts required. Because COQ10 shares a biosynthetic pathway with cholesterol, those patients on statins would be benefited with supplementation using the phospholipid and COQ10, which is found in many membranes of organelles, including mitochondrion. COQ10 is fat-soluble and the phospholipids enhance absorption. COQ10 also operates as an antioxidant from its energy carrier function and may accept electrons to be reduced. When it gives up electrons, it is oxidized. COQ10 has a similar absorption as that of lipids with secretion into the small intestines of pancreatic enzymes and bile that facilitates emulsification and micell formation as required for absorption of lipophilic substances. It is possible to reduce the overall particle size to improve bioavailability using nano-particles. It is possible to use an emulsion system to facilitate absorption from the gastrointestinal tract and improve bioavailability such as using lipid microspheres, including emulsions from soybean oil and lecithins for stabilization. It is possible to modify the COQ10 to increase solubility of water and facilitate absorption. This may include use of a tyloxapol polymer and other solubilising agents such as hydrogenated lecithin. It is possible to use other nano-particles and dendrimers or liposomes to enhance bioavailability of the COQ10.

It has now been determined that lower dosages of astaxanthin may be used when it is added with at least one of a phospholipid, glycolipid, and sphingolipid, and preferably greater concentrations of phospholipid. It is believed the phospholipid makes the substrate much more bioavailable. A pharmaceutical or food grade diluent may be added or other surfactant. The phospholipids may include plant based phospholipids such as from lecithin and lysophospholipids and/or glycophospholipids, including the addition of a seed oil extract, such as perilla. Astaxanthin levels could very from 0.5-2 mg and 0.5-4 mg and in one embodiment is 2-4 mg or 2-6 mg and as broad as 0.5-8 mg and 0.5-12 mg and 7-12 mg.

In induced uveitis, astaxanthin also showed dose dependant ocular anti-inflammatory activity by suppression of NO, PGE-2 and TNF-Alpha by directly blocking NO synthase activity. As noted before, astaxanthin is also known to reduce C-Reactive Protein (C-RP) blood levels in vivo. For example, in human subjects with high risk levels of C-RP three months of astaxanthin treatment resulted in 43% of patients serum C-RP levels to drop below the risk level. Astaxanthin is absorbed in vitro by lens epithelial cells where it suppresses UVB induced lipid peroxidative mediated cell damage at umol/L concentrations. In human trials astaxanthin at 4 mgs/day prevented post exercise joint fatigue following strenuous knee exercise when compared to untreated subjects. At 15 mg a day, it has been known to treat osteoarthritis.

Astaxanthin has potent singlet oxygen quenching activity. Astaxanthin typically does not exhibit pro-oxidant activity unlike β-carotene, lutein, zeaxanthin and Vitamins A and E. Astaxanthin in some studies has been found to be about 50 times more powerful than Vitamin E, 11 times more powerful than β-carotene and three times more powerful than lutein in quenching of singlet oxygen. Astaxanthin is also well known for its ability to quench free radicals. Comparative studies have found astaxanthin to be 65 times more powerful than Vitamin C, 54 times more powerful than β-carotene, 47 times more powerful than lutein, and 14 times more powerful than Vitamin E in free radical quenching ability.

The '533 Tso Patent above discloses the benefits of astaxanthin for retarding and ameliorating central nervous system and eye damage. Astaxanthin crosses the blood-brain-retina barrier and this can be measured by direct measurement of retinal astaxanthin concentrations. Thus, Tso demonstrated protection from photon induced damage of photo-receptors, ganglion and neuronal cell damage.

It is now known that oils having an oil based phospholipid bound EPA and DHA uptake into cellular membranes is far more efficient than triacylglyercide bound EPA and DHA, since liver conversion of triacylglycerides is itself inefficient and because phospholipid bound EPA and DHA can be transported into the blood stream via the lympathic system, thus, avoiding liver breakdown.

The current composition, such as for eye or cardiovascular health, can be delivered advantageously for therapeutic results with 1-4,000 mg of oil, such as a seed based oil with or without added phospholipids, and delivered per daily dose. In another example, 500 mg is a preferred amount for a single capsule dosage, and in another example 1,000 mg. Ranges of 500 to 1,000 mg are possible, including 750 mg, including with an added phospholipid. In another example, 0.1-50 mg astaxanthin are supplemented to the oil per daily dose, but an example amount includes about 2-4 mg and 0.5 to 12 mg or 0.5 to 8 mg. The phospholipid based oils may include roe extract with phospholipid and plant based oils and other phospholipids. It is possible to also use wax esters and omega-3 salts and ethyl esters.

The composition may also include an n-3 (omega-3) fatty acid rich oil derived from different oils, including fish oil, algae oil, flax seed oil, or chia seed oil when the n-3 fatty acid comprises alpha-linolenic, stearidonic, eicosapentaenoic or docosapentaenoic acid. The composition may include naturally-derived and synthetic antioxidants that are added to retard degradation of fatty acids and astaxanthin.

Details of a type of CO2 extraction and processing technology (as supercritical CO2 extraction) and peroxidation blocker technology that can be used are disclosed in commonly assigned U.S. Pat. No. 8,652,544; U.S. Pat. No. 8,586,104; U.S. Pat. No. 8,784,904; and U.S. Patent Publication No. 2009/0181114, the disclosures which are hereby incorporated by reference in their entirety.

In one example, about 150 mg to about 300 mg of perilla or other oil extract and/or other oils and phospholipids are used. In another example, 2 to 4 mg astaxanthin are supplemented to the seed oil extract and/or phospholipids and may include a range of 0.5 to 4 mg, or 0.5 to 6 mg, 0.5 to 8 mg, 0.5 to 12 mg, or 7 to 12 mg, and other ranges as described before.

The perilla and/or other seed oil extract may be prepared by using supercritical CO2 and/or solvent extractions for selective removal of triacylglycerides from phospholipids such as using the techniques in the incorporated by reference patents. The composition, such as for eye care or cardiovascular care, may also include a natural or synthetic cyclooxygenase-1 or -2 inhibitor comprising for example aspirin, acetaminophen, steroids, prednisone, or NSAIDs. The eye care composition may also include a gamma-linoleic acid rich oil comprising Borage (Borago officinalis L.) or Safflower (Carthamus tinctorius L.), which delivers a metabolic precursor to PGE₁ synthesis.

The composition whether for eye care or cardiovascular or other care may also include an n-3 (omega-3) fatty acid rich oil derived from fish oil, algae oil, flax seed oil, chia seed oil or perilla seed oil wherein the n-3 fatty acid source comprises alpha-linolenic, stearidonic, eicosapentaenoic or docosapentaenoic acid. The eye care composition may include naturally-derived and synthetic antioxidants that are added to retard degradation of fatty acids such as tocopherols, tocotrienols, carnosic acid or Carnosol and/or astaxanthin.

It has been found advantageous to use herring roe extract as the source of phospholipids that may have some EPA and DHA with the carotenoids either alone or in combination with the seed oil extract. Other phospholipids without EPA and DHA may be used. Sources include egg yolk extract and seed oil phospholipids. Synergistic results are obtained and vast improvements seen. One study indicated that phospholipids from herring roe improved phospholipid and glucose tolerance in healthy, young adults as published by Bjorndal et al., Lipids in Health Disease, 2014, 13:82. The pure roe phospholipid may be formed using extraction techniques. It is a honey-like product that is thinned or diluted with fish oil and/or perilla oil or other seed or plant oil, and in an example, with the perilla oil as described above.

The specification prior to dilution with fish oil and/or perilla oil is as follows:

| | |
|---|---|
| Percentage that is phospholipids | 60 |
| Phospholipid mg/g | 600 |
| Phosphatidyl choline portion mg/g | 520 |
| Choline equivalents | 83 |
| Total EPA mg/g (TG & PL bound) | 75 |
| Total DHA mg/g (TG & PL bound) | 195 |
| EPA mg/g bound to phospholipid | 67 |
| DHA mg/g bound to phospholipid | 175 |
| EPA + DHA mg/g bound to phospholipid | 242 |

The herring roe extract is processed in one example using extraction by ethanol. Triacylglycerides are added and ethanol stripped out to have a robust solution. Seed oil, such as the perilla seed oil as described in the incorporated by reference '904 patent, may be added back to the ethanol extract before stripping to thin and form a high level phospholipid blend. The roe oil extract may be mixed with fish oil and/or seed oil, such as the perilla, or any other oil, including a marine one. In an example, the herring egg roe extract is mixed with seed oil of at least 1:1 and with perilla preferably as high as 6:1 ALA to LA with the concentrate as having at least 50%, and in another example 60% phospholipids, and in another example at least 30%, and in another example 40% triglycerides. This is a non-limiting example.

An example composition for eye or cardiovascular health includes a combination of a roe extract from herring or a phospholipid rich roe extract with phospholipid bound EPA and DHA admixed with seed/fish oil and/or seed oil where the seed oil has a ratio of ALA to LA between 1:1 and 1:6, and optionally including astaxanthin in one example of about 2 to 4 mg, or 0.5 to 8 mg, or 0.5 to 12 mg, or other ranges as noted above. The amount of roe egg extract mixed with the seed oil such as perilla oil varies and is about 150 to 500 mg, or 300 to 500 mg, or up to 1,000 mg daily dose in one example, and includes the astaxanthin and the lutein and trans-zeaxanthin or meso-zeaxanthin. Other plant based phospholipids may be used, including commercially available lecithins and an egg yolk derivative, including lysophospholipids and glycophospholipids to act as surfactants. It is possible to use sunflower-based phospholipids and natural plant-based oils and natural surfactant extracts. Phospholipids may be derived from egg yolk and seed oil phospholipids, including sunflower, safflower and soybean. The astaxanthin is enhanced with fats, surfactants, or phospholipids and can be delivered more efficiently with phospholipids and sunflower based and/or the lipophilic perilla oil as described before.

In an example, the composition is encapsulated into a single dosage capsule. In a specific example, the encapsulated composition includes the carotenoids as explained above and seed oil extract such as from perilla and/or herring caviar phospholipid extract (herring roe) perilla (perilla frutescens) seed extract, olive oil, Zanthin® astaxanthin (Haematococcus pluvialis algae extract), gelatin, spice extract, non-GMO natural tocopherols, cholecalciferol, riboflavin, and methylcobalamin. The composition may include fish as herring roe and tilapia gelatin. An example is set forth in the following chart.

### Properties:

| | |
|---|---|
| Appearance | Size 00 clear capsule with dark red |
| | oily fill |
| Fatty Acids | |
| ALA | min. 140 mg |
| EPA | min. 18 mg |
| DHA | min. 50 mg |
| Total Omega-3 | min. 210 mg |
| Phospholipids | 195 mg |
| Astaxanthin | 500 μg |
| Vitamin D₃ | 1000 IU; 250% DV |
| Vitamin B₂ (Riboflavin) | 1.7 mg; 100% DV |
| Vitamin B₁₂ | 6 μg; 100% DV |
| Microbiological | USP <61>/FDA BAM |
| Total Plate Count | <1000 cfu/g |
| Yeast & Mold | <100 cfu/g |
| E. coli | Absent in 10 g |
| Salmonella | Absent in 10 g |
| S. aureus | Absent in 10 g |
| Storage | |
| Conditions | Tightly closed containers, |
| | 15-30° C., |
| | 30-50% RH |
| Shelf-life | 24 months minimum |
| Packaging | HDPE or PET bottle (count TBD) |

All ingredients BSE-free and non-GMO

The processing components may contain a mix of marine omega-3 phospholipids derived from herring caviar and perilla seed oil. It may contain an O2B™ botanical peroxidation blocker as described above, including spice extract, non-GMO tocopherols and ascorbyl palmitate. It can be packaged as a bulk product in sealed drums 45 and 190 kg net with inert headspace, complying with European and American standards for food products. It preferably stores at below room temperature. The product is protected against light and heat. If drums are opened for sampling, the headspace can be flushed with inert gas during sampling and prior to storing.

| Test | Unit | Acceptance Criterion | | Method |
|---|---|---|---|---|
| Appearance | | Amber viscous oil | | AM2020 |
| Solubility | | Oil soluble and water | | AM2021 |
| | | dispersible | | |
| | | Minimum | Maximum | |
| ALA (C18:3 n-3) | mg/g as TG³⁾ | 230 | | AM1044 |
| EPA (C20:5 n-3) | mg/g as TG³⁾ | 30 | | AM1001 |
| DBA (C22:6 n-3) | mg/g as TG³⁾ | 85 | | AM1001 |
| Total omega-3 ¹⁾ | mg/g as TG³⁾ | 370 | | AM1001 |
| ALA (C18:3 n-3) | mg/g as FFA⁴⁾ | 215 | | AM1044 |
| EPA (C20:5 n-3) | mg/g as FFA⁴⁾ | 28 | | AM1001 |
| DHA (C22:6 n-3) | mg/g as FFA⁴⁾ | 80 | | AM1001 |
| Total omega-3 ¹⁾ | mg/g as FFA⁴⁾ | 335 | | AM1001 |
| Total PC | mg/g | 250 | | AM1002 |
| Total PL | mg/g | 300 | | AM1002 |
| Total neutral lipids | mg/g | | 700 | AM1003 |
| Water content by | % | | 3.0 | AM1004 |
| Karl Fisher | | | | |
| Peroxide value | meq/kg | | 10.0 | AM1005 |
| Heavy metals (sum | mg/kg | | 10 | AM1015 |
| of Pb, Hg, Cd & | | | | |
| In-organic As) ²⁾ | | | | |

¹⁾ Total n-3: ALA, EPA, DHA, 18:4, 20:4, 21:5, 22:5
²⁾ Frequency analysis
3)All ALA, EPA, DHA or Total omega-3 expressed as triglycerides
⁴⁾All ALA, EPA, DHA or Total omega-3 expressed as free fatty acids

As noted before, it has been surprisingly found that the astaxanthin may be made more bioavailable when incorporated or used with one of at least a phospholipid, glycolipid, and sphingolipid and optionally with food and/or pharmaceutical grade diluents. The lutein is also made more bioavailable and this may work in conjunction with the seed oil extract such as from perilla. In an example, the astaxanthin may be about 0.1 to 15 percent by weight of the at least one phospholipid, glycolipid, and sphingolipid and any seed oil extract. The astaxanthin in an example is derived from a natural or synthetic ester or natural or synthetic diol. A pharmaceutical or food grade diluent may be added. Instead of astaxanthin, lutein may be used alone with the phospholipid. COQ10 may be used.

It should be understood that the triglycerides have two types of molecules as a glycerol and three fatty acids, while the phospholipids contain glycerol and fatty acids, but have one glycerol molecule and two fatty acid molecules. In place of that third fatty acid, a polar group is instead attached to the glycerol molecule so that the phospholipids are partly hydrophilic as compared to hydrophobic triglycerides. Lysophospholipids may be used as a derivative of a phospholipid in which one or both acyl derivatives have been removed by hydrolysis. Lecithin and its derivatives may be used as an emulsifier and surfactant as a wetting agent to reduce surface tension of liquids. Other phospholipids may be used. Different phospholipids include phosphatidylcholine, phosphatidylethanolamine, phosphatidylserine, phosphatidylinositol, phosphatidic acid, lyso-phosphatidylcholine, lyso-phosphatidylethanolamine, and lyso-Phosphatidylserine. Some may be derived from egg yolk and extracted chemically using hexane, ethanol, acetone, petroleum ether or benzene, and also extracted mechanically, including from different sources such as soybeans, eggs, milk, marine sources, sunflower and safflower. When derived from soya and sunflower, phospholipids may include those products mentioned before, including phosphatidic acid. Various compositions such as lecithin may be hydrolyzed enzymatically and have a fatty acid removed by phospholipase to form the lysophospholipids that can be added to the roe extract as explained above. One phospholipase is phospholipase A2 where the fatty acid is removed at the C2 position of glycerol. Fractionation may be used to enhance the phospholipids.

The glycolipids are primarily derivatives of ceramides where a fatty acid is bonded or connected to the amino alcohol sphingosine. It should be understood that the phospholipid sphingomyelin is also derived from a ceramide. Glycolipids, however, contain no phosphates in comparison to the phospholipids. The fat is connected to a sugar molecule in a glycolipid and are fats bonded to sugars. Because it is built from a sphingosine, fat and sugar, some refer to it as a glycosphingolipid. A sphingolipid is a lipid that contains a backbone of sphingoid basis and set of alphatic amino alcohols that include the sphingosine.

As noted before, the phospholipid and other components may be derived from at least one of a plant, algae and animal source, or a synthetic derivative thereof. The phospholipid and other components may be derived from at least one of soybean, sunflower, safflower, grapeseed, egg yolk, krill, fish body, fish roe, squid, and algae. The phospholipid and other components may be formed as compound rich mono- or di-glcerides or fatty acids where the fatty acid contains between 2 and 20 carbon atoms. During processing, the composition may be formed by dispersing the astaxanthin and other carotenoids and phospholipid and optionally a diluent under high shear conditions. The diluent may be a pharmaceutical or food grade diluent as known to those skilled in the art.

In another example, the astaxanthin is about 2 to about 10 percent by weight of the seed oil extract and/or phospholipid. It may include the glycolipid and derived from a natural or synthetic ester or synthetic diol. In yet another example, 50 to 500 mg or 50 to 1,000 mg of phospholipid and any added glycolipid and sphingolipid may be used together with the carotenoids. The dietary supplement composition may be formulated into a single dosage capsule.

It is possible to also add and use a shrimp oil that is extracted from shrimp mince and shells that include high levels of phospholipids and omega-3 fatty acids such as EPA and DHA. The oils may be rich in astaxanthin. The parts per million of astaxanthin can range from 3,000 to 7,000, and in one example, from 3,500 to 6,000 parts per million. Total phospholipids may be about 25-35% w/w and higher up to 50% and 60%. The total omega-3 may be greater than about 16.0 grams per 100 grams with an EPA percentage of greater than 17% and about greater than 8 grams per 100 grams of the DHA percent of greater than 14. These ranges can vary, of course.

It is possible to add to this composition some low molecular weight hyaluronic acid, such as derived from microbial fermentation and other sources, including hydrolyzed animal tissues, and could range from 0.5 to 300 kDa or 0.5 to 100 kDa. It could be derived from chicken sternal cartilage extract. The hyaluronic acid may include elastin, elastin precursors, and collagen. The hyaluronic acid may be contained in a matrix form with chondroitin sulfate and naturally occurring hydrolyzed collagen Type II nutraceutical ingredients and form lower weight molecules that the body may more readily absorb and deliver to different areas of the body as required. Fresh chicken sternal cartilage could be cut and suspended in aqueous solution followed by treating the cartilage with a proteolytic enzyme to form a hydrolysate. The proteolytic enzyme is capable of hydrolyzing collagen Type II to fragments having a lower molecular weight. The hydrolysate is sterilized and filtered and concentrated and then dried to form powder enriched collagen Type II powder that is then isolated and includes a percentage of low molecular weight hyaluronic acid. Examples of manufacturing techniques can be found in U.S. Pat. Nos. 6,780,841 and 6,025,327, the disclosures which are hereby incorporated by reference in their entirety. It is possible that the low molecular weight hyaluronic acid could also be derived from the hydrolyzed collagen as derived from the bovine collagen Type I or the chicken sternal cartilage collagen Type II, or even a natural eggshell membrane that includes some hyaluronic acid, which can be extracted from the eggshell membrane.

It is also possible to use a pure diol of the S, S′astaxanthin, including a synthetic diol with the surfactant such as the phospholipid and/or seed oil extract such as from perilla with the other carotenoids. It is possible to use that pure diol in combination with an EPA rich algae based oil or other fish, roe extract, or plant based oil and/or phospholipid and/or surfactant as described above, and which is admixed with either astaxanthin derived from Haematococcus pluvialis or the free diol form in substantially pure S,S′ enantiomer form. It can be mixed with the CQ10 or lutein alone. It is possible to add synthetically derived mixed enantiomers of the diol. The diol of the S, S′astaxanthin is possible because in cases of krill oil and possibly algae based oils and Hp derived and other types, there are principally diesters and monoesters respectively with very little diol, which is insoluble. Some research indicates that it may be many times more bioavailable than either the monoester or diester form. It is possible to synthesize asymmetrically the S,S′ pure diol. Despite the pure diol's poor solubility in some examples, there may be an active transport mechanism related to its bioavailability, or conversely, that only in the diol form is the monoester or diester forms transferred from the intestines to the blood. The phospholipids may have added glycolipids present with EPA and/or DHA along with the added astaxanthin and other carotenoids in its various forms and especially the S,S′ enantiomeric form in principally monoester form from Haematococcus pluvialis or pure diol form from asymmetric synthesis could be more viable for the composition. Thus, it is possible to combine it with the algae derived glycol and phospholipid based EPA rich oil.

As noted before, astaxanthin (3,3′-dihydroxy-β-β-carotene-4,4′-dione) is a xanthophyll carotenoid found in many marine species including crustaceans, salmonid fish and algae. Astaxanthin cannot be synthesized by mammals, but when consumed in the diet has shown effectiveness as an antioxidant, anti-inflammatory agent and with benefit to eye health, heart health, and the immune system.

Astaxanthin has a hydroxyl group on each β-ionone moiety, therefore it can be found in its free (diol) form as well as mono- or di-esterified. In natural products astaxanthin is commonly found as a mixture: primarily mono-esters of C12-C18 fatty acids and lesser amounts of di-ester and free diol. Synthetic astaxanthin is commonly provided in only the free diol form.

The astaxanthin molecule has two E/Z chiral centers and three optical R/S isomers. Haematococcus pluvialis algae produces natural astaxanthin solely in the (3S,3′S) isomer. This is explained in the article from Renstrøm B., G. Borch, O. Skulberg and S. Liaane-Jensen, “Optical Purity of (3S,3′S) Astaxanthin From Haematococcus Pluvialis,” Phytochemistry, 20(11): 2561-2564, 1981, the disclosure which is hereby incorporated by reference in its entirety.

Alternatively, the yeast Phaffia rhodozyma synthesizes only the 3R,3′R configuration. This is explained in the article from Andrewes A. and M. Starr entitled, “(3R,3′R)-Astaxanthin from the Yeast Phaffia Rhodozyma,” Phytochemistry, 15:1009-1011, 1976, the disclosure which is hereby incorporated by reference in its entirety.

Wild salmon predominately contain the (3S,3′S) form with a (3S,3′S), (3R,3′S), and (3R,3′R) isomer ratio of 22:1:5. This is explained in the article from Turujman, S, W. Warner, R. Wei and R. Albert entitled, “Rapid Liquid Chromatographic Method to Distinguish Wild Salmon From Aquacultured Salmon Fed Synthetic Astaxanthin,” J. AOAC Int., 80(3): 622-632, 1997, the disclosure which is hereby incorporated by reference in its entirety.

However, astaxanthin produced by traditional synthesis will contain a racemic mixture in a (3S,3′S), (3R,3′S; meso), (3R,3′R) ratio of 1:2:1. This ratio is also seen in many species of shrimp, which are able to racemize (3S,3′S) to the meso form. This is explained in the article from Schiedt, K., S. Bischof and E. Glinz entitled, “Metabolism of Carotenoids and in vivo Racemization of (3S,3′S)-Astaxanthin in the Crustacean Penaeus,” Methods in Enzymology, 214:148-168, 1993, the disclosure which is hereby incorporated by reference in its entirety.

However, most of the astaxanthin in shrimp is within the carapace (shell) therefore limited amounts of the meso isomer are consumed in the human diet.

Feeding studies of free diol or fatty acid esters of astaxanthin has been shown to increase the amount of astaxanthin in human plasma. This is explained in the article from Østerlie, M., B. Bjerkeng and S. Liaan-Jensen, entitled “Plasma Appearance and Distribution of Astaxanthin E/Z and R/S Isomers in Plasma Lipoproteins of Men After Single Dose Administration of Astaxanthin,” J. Nutr. Biochem, 11:482-490, 2000; and the article from Coral-Hinostroza, G., T. Ytestøyl, B. Ruyter and B. Bjerkeng entitled, “Plasma Appearance of Unesterified Astaxanthin Geometrical E/Z and Optical R/S Isomers in Men Given Single Doses of a Mixture of Optical 3 and 3′R/S Isomers of Astaxanthin Fatty Acyl Diesters,” Comp. Biochem Phys. C., 139:99-110, 2004, the disclosures which are hereby incorporated by reference in their entirety.

The uptake of free astaxanthin diol is about 4-5 times higher than that of esterified astaxanthin, likely due to the limitation of required enzymatic hydrolysis in the gut prior to absorption. These intestinal enzymes may also be R/S selective on astaxanthin esters. Coral-Hinostroza et al. (2004) found higher relative absorption of astaxanthin from (3R,3′R-astaxanthin dipalmitate compared to the other two isomers. However, ingestion of racemic free diol astaxanthin does not show any stereospecific selection.

Astaxanthin for use in human food supplements is currently derived in an example from the cultivated freshwater algae Haematococcus pluvialis. This algae produces 3S,3′S astaxanthin ester in a fatty acid matrix which can be isolated with solvent or carbon dioxide extraction. This oily extract can be used directly in edible formulations or further processed into solid powder or beadlet preparations. Many clinical studies have been conducted with H. pluvialis derived astaxanthin to demonstrate beneficial health effects and safety. Food additive approvals for astaxanthin-rich algae extracts have been approved for many suppliers in the US and EU.

Haematococcus algae cultivation for use in dietary supplements cannot always match demand for use of astaxanthin in dietary supplements. Use of synthetic astaxanthin diol can also benefit applications which need a concentrated, standardized astaxanthin source. Conventional racemic synthetic astaxanthin sources are used as a colorant in Salmonid aquaculture as a feed ingredient. This racemic mixture may have limited use since only one-quarter of the compound is the 3S,3′S isomer commonly found in natural Salmon and has been studied in humans for efficacy and safety.

Astaxanthin may also be synthesized with in a stereospecific manner, so that the output is exclusively the generally accepted 3S,3′S isomer in a free diol form. The free diol crystals can be suspended in a vegetable oil or solid beadlet for use in edible preparations or pill, capsule, tablet form. The 3S,3′S product has the advantage of greater consistency than algal preparations and also with lower odor. Therefore algal-derived astaxanthin can be replaced with synthetic 3S,3′S astaxanthin diol in existing formulations with the same or increased effectiveness.

It is also known that algae can be an important source for omega-3 fatty acids such as EPA and DHA and can be used in combination with phospholipids and other carotenoids and other components for the composition for eye or cardiovascular health, and can include the seed oil extracts and/or phospholipids as described above. It is known that fish and krill do not produce omega-3 fatty acids but accumulate those fatty acids from the algae they consume. Omega-3 bioavailability varies and is made available at the site of physiological activity depending on what form it is contained. For example, fish oil contains omega-3 fatty acids in a triglyceride form that are insoluble in water and require emulsification by bile salts via the formation of micelles and subsequent digestion by enzymes and subsequent absorption. Those omega-3 fatty acids that are bound to polar lipids, such as phospholipids and glycolipids, however, are not dependent on bile for digestion and go through a simpler digestion process before absorption. Thus, these omega-3 fatty acids, such as from an algae based oil, have greater bioavailability for cell growth and functioning as compared to the omega-3 triglycerides of fish oil and can be used with the carotenoids and other components in some examples for the eye care composition. There are many varieties of algae that contain EPA conjugated with phospholipid and glycolipid polar lipids or contain EPA and DHA conjugated with phospholipids and glycolipids.

Throughout this description, the term “algae” or “microalgae” may be used interchangeably to each other with microalgae referring to photosynthetic organisms that are native to aquatic or marine habitats and are too small to be seen easily as individual organisms with the naked eye. When the term “photoautotropic” is used, it refers to growth with light as the primary source of energy and carbon dioxide as the primary source of carbon. Other forms of biomass that may encompass algae or microalgae may be used and the term “biomass” may refer to a living or recently dead biological cellular material derived from plants or animals. The term “polar” may refer to the compound that has portions of negative and/or positive charges forming negative and/or positive poles. The term “oil” may refer to a combination of fractionable lipid fractions of a biomass. As known to those skilled in the art, this may include the entire range of various hydrocarbon soluble in non-polar solvents and insoluble, or relatively insoluble in water as known to those skilled in the art. The microalgae may also include any naturally occurring species or any genetically engineered microalgae to have improved lipid production.

Algae based oil may be used in addition to the seed oil extract and/or phospholipids with the carotenoids in the composition, in an example, and supplemented to the seed oil extract such as from perilla. This algae based oil may provide an algae sourced EPA or an EPA/DHA based oil in which oils are present in phospholipid and glycerolipid forms, as glycolipids. Different algae based oils derived from different microalgae may be used. One preferred example algae based oil has the EPA titre higher than the DHA as compared to a class of omega-3's from fish oils that are triacylglycerides. These algae based oils are rich in EPA and in the phospholipid and glycolipid forms. An example marine based algae oil is produced by Parry Nutraceuticals as a division of EID Parry (India) Ltd. as an omega-3 (EPA) oil.

The following first table shows the specification of an algae based oil as manufactured by Parry Nutraceuticals identified above, followed by a second table for a fatty acid profile chart of that algae based oil. A third table is a comparative chart of the fatty acid profiles for non-algae based oils. These charts show that the algae based oil has a high EPA content of phospholipids and glycolipids. The algae based oils may be processed to enrich selected constituents using supercritical CO2 and/or solvent extractions as noted above and other techniques.

### Specification: Algae Based Oil

| | SPECIFI- | SOP. | TEST METHOD/ |
|---|---|---|---|
| PARAMETERS | CATION | NO | REFERENCE |
| Physical Properties | | | |
| Appearance | Viscous oil | QA-88 | In house |
| Color | Brownish black | QA-88 | In house |
| Odor | Characteristic | QA-88 | In house |
| Taste | Characteristic | QA-88 | In house |
| General Composition | | | |
| Loss on drying (%) | 2.0-3.0 | QA-038 | USP <731> Loss |
| | | | on drying |
| Ash (%) | 0.5-1.0 | QA-080 | AOAC Official |
| | | | Method |
| | | | 942.05, 16th |
| | | | Edition |
| Protein (%) | 1.0-2.0 | QA-021 | AOAC Official |
| | | | method 978.04, |
| | | | 16th Edn, |
| Carbohydrate (%) | 1.0-2.0 | | AOAC 18th Edn |
| | | | 2006/By |
| | | | Difference |
| Residual Solvent (ppm) | NMT 100 | QA-074 | GC-Head |
| (as Ethyl Acetate) | NMT 30 | | Space, |
| (as Acetone) | | | USP < 467) |
| Lipid Composition | | | |
| Total Lipid (%) | 92.0-95.0 | QA-86 | AOAC official |
| | | | method 933.08 |
| Chlorophyll (%) | NMT 1.50 | QA-078 | Jeffrey & |
| | | | Humphrey (1975)- |
| | | | Photosynthetic |
| | | | pigments of |
| | | | Algae (1989) |
| Total carotenoids (%) | NMT 1.50 | QA-85 | By JHFA |
| | | | method-1986 |
| Total Unsaponifiables (%) | NMT 12.0 | QA-086 | AOAC official |
| | | | method 933.08 |
| Omega 3 [EPA + DHA]- | NLT 15.00 | QA-087 | In House method |
| % w/w | | | |
| Total Omega 3 (% w/w) | NLT 17.00 | | |
| Total Omega 6 (% w/w) | NMT 5.00 | | |
| Total EFA (% w/w) | NLT 20. | | |
| Lipid percentage | | | |
| Triglycerides | 15-20% | | |
| Phospholipids | 5-10% | | |
| Glycolipids | 35-40% | | |
| Free fatty acids | 15-20% | | |
| Microbial parameters | | QA-039 | AOAC, 1995, |
| Standard Plate Count | NMT 1,000 | | Chapter 17 |
| (cfu/1 g) | | | |
| Yeast & Mold (cfu/1 g) | NMT 100 | | |
| Coli forms (/10 g) | Negative | | |
| E. Coli (/10 g) | Negative | | |
| Staphylococcus (/10 g) | Negative | | |
| Salmonella (/10 g) | Negative | | |
| Fatty acid profile (Area %) | | | |
| Myristic acid [14.0] | NLT 4.0 | QA-086 | In House GC |
| Palmiltic acid [16:0] | NLT 16.0 | & 087 | method |
| Palmito oleic acid | NLT 12.0 | | |
| [16:1, n-9] | | | |
| Hexadecadienoic acid | NLT 4.0 | | |
| [16:2, n-4] | | | |
| Hexadecatrienoic acid | NLT 12.0 | | |
| [16:3, n-4] | | | |
| Stearic acid [18:0] | NLT 0.10 | | |
| Oleic acid [18:1] | NLT 1.0 | | |
| Linoleic acid [18:2, n-6]- | NLT 1.0 | | |
| LA | | | |
| AlphaLinolenic acid | NLT 0.50 | | |
| [18:3, n-3]-ALA | | | |
| Stearidonic acid [18:4, | NLT 0.10 | | |
| n-3]-SA | | | |
| Arachidonic Acid [20:4, | NLT 0.25 | | |
| n-6)-AA | | | |
| Eicosapentaenoic acid | NLT 15.0 | | |
| [20:5, n-3] | | | |
| Decosahexaenoic acid | NLT 1.5 | | |
| [20:6, n-3] | | | |
| Heavy Metals | | | |
| Lead (ppm) | NMT 1.0 | External | AOAC 18th |
| Arsenic (ppm) | NMT 0.5 | lab | Edn: 2006 By |
| Cadmium (ppm) | NMT 0.05 | reports | ICPMS |
| Mercury (ppm) | NMT 0.05 | | |

Safety: Safe for the intended use
Shelf life: 24 months from the date of manufacture
Stability: Stable in unopen conditions
Storage: Store in a cool, dry place away from sunlight, flush container with Nitrogen after use
Documentation: Every Batch of shipment carries COA
Packing: 1 kg, 5 kg, and 20 kg food grade containers

### Fatty Acid Profile Chart

### Algae Based Oil

| | ALGAE BASED |
|---|---|
| | OMEGA-3 |
| FATTY ACID | (EPA) OIL |
| Total fatty acid, gm/100 gm of oil | 75 gm |
| Fatty acid [% of total fatty acid] | |
| Myristic acid [14:0] | 6.87 |
| Pentadecanoic acid [15:0] | NA |
| Palmitic acid [16:0] | 20.12 |
| Palmito oleic acid [16:1, ω-9] | 18.75 |
| Hexadeca dienoic acid [16:2, ω-4] | 6.84 |
| Hexadeca trienoic acid [16:4, ω-4] | 12.54 |
| Heptadecanoic acid [17:0] | NA |
| Stearic acid [18:0] | 0.68 |
| Oleic acid [18:1, ω-9] | 3.56 |
| Linoleic acid [18:2, ω-6] | 2.68 |
| Alpha linolenic acid [18:3, ω-3] | 3.73 |
| Gamma linolenic acid [18:3, ω-6] | NA |
| Stearidoni acid [18:4, ω-3] | 0.33 |
| Arachidonic acid [20:4, ω-6] | 0.97 |
| Eicosapentaenoic acid [20:5, ω-3] EPA | 23.00 |
| Docosapentaenoic acid [22:5, ω-3] DHA | NA |
| Docosahexaenoic acid [22:6, ω-3] DHA | 3.26 |
| others | 3.54 |
| EPA/DHA [gm/100 gm oil] | 15.75 |
| Total ω-3 fatty acids [gm/100 gm oil] | 18.20 |
| LIPD CLASS DETAILS [gm/100 gm oil] | |
| Unsaponifiables [carotenoids, chlorophyll, | 12 |
| sterol, fatty alcohol etc.,] | |
| Free fatty acids | 20 |
| Triglydcerides | 20 |
| Phospholipids | 10 |
| Glycolipids | 38 |
| Total | 100 |
| STABILITY [months] | 24 |

### Fatty Acid Profile—Comparative Chart

### Non-Algae Based Oils

| | FISH OIL | KRILL | MARTEK |
|---|---|---|---|
| FATTY ACID | MAXEPA | OIL | OIL |
| Total fatty acid, gm/100 gm of oil | 95 gm | 70-80 gm | 95 gm |
| Fatty acid [% of total fatty acid] | | | |
| Myristic acid [14:0] | 8.68 | 11.09 | 11.47 |
| Pentadecanoic acid [15:0] | NA | NA | NA |
| Paimitic acid [16:0] | 20.35 | 22.95 | 26.36 |
| Palmito oleic acid [16:1, ω-9] | 11.25 | 6.63 | NA |
| Hexadeca dienoic acid [16:2, ω-4] | NA | NA | NA |
| Hexadeca trienoic acid [16:4, ω-4] | NA | NA | NA |
| Heptadecanoic acid [17:0] | NA | NA | NA |
| Stearic acid [18:0] | 4.67 | 1.02 | 0.50 |
| Oleic acid [18:1, ω-9] | 13.07 | 17.93 | 1.50 |
| Linoleic acid [18:2, ω-6] | 1.28 | 0.14 | 0.61 |
| Alpha linolenic acid [18:3, ω-3] | 0.33 | 2.11 | 0.40 |
| Gamma linolenic acid [18:3, ω-61 | NA | NA | NA |
| Stearidonic acid [18:4, ω-3] | 1.69 | 7.01 | 0.33 |
| Arachidonic acid [20:4, ω-6] | 0.50 | NA | NA |
| Eicosapentaenoic acid [20:5, ω-3] | 20.31 | 19.04 | 1.0 |
| EPA | | | |
| Docosapentaenoic acid [22:5, ω-3] | NA | NA | 15.21 |
| DHA | | | |
| Docosahexaenoic acid [22:6, ω-3] | 13.34 | 11.94 | 42.65 |
| DHA | | | |
| others | 4.53 | 0.14 | NA |
| EPA/DEA [gm/100 gm oil] | 31.96 | 21.68 | 41.46 |
| Total ω-3 fatty acids [gm/100 gm | 33.85 | 28.00 | 41.60 |
| oil] | | | |
| LIPD CLASS DETAILS | | | |
| [gm/100 gm oil] | | | |
| Unsaponifiables | 5 | 5 | 5 |
| [carotenoids, chlorophyll, sterol, | | | |
| fatty alcohol etc.,] | | | |
| Free fatty acids | 0.5 | 30 | 0.5 |
| Triglydcerides | 94.5 | 25 | 94.5 |
| Phospholipids | Nil | 40 | Nil |
| Glycolipids | Nil | Nil | Nil |
| Total | 100 | 100 | 100 |
| STABILITY [months] | 12 | 24 | 6 |

Different types of marine based algae oils may be used, including nannochloropsis oculata as a source of EPA. Another algae that may be used is thalassiosira weissflogii such as described in U.S. Pat. No. 8,030,037 assigned to the above-mentioned Parry Nutraceuticals, a Division of EID Parry (India) Ltd., the disclosure which is hereby incorporated by reference in its entirety. Other types of algae as disclosed include chaetoceros sp. or prymnesiophyta or green algae such as chlorophyta and other microalgae that are diamons tiatoms. The chlorophyta could be tetraselmis sp. and include prymnesiophyta such as the class prymnesiophyceae and such as the order isochrysales and more specifically, isochrysis sp. or pavlova sp.

There are many other algae species that can be used to produce EPA and DHA as an algae based oil whether marine based or not to be used in accordance with a non-limiting example. In some cases, the isolation of the phospholipid and glycolipid bound EPA and DHA based oils may require manipulation of the algae species growth cycle.

Other algae/fungi phospholipid/glycolipid sources include: grateloupia turuturu; porphyridium cruentum; monodus subterraneus; phaeodactylum tricornutum; isochrysis galbana; navicula sp.; pythium irregulo; nannochloropsis sp.; and nitzschia sp.

Details regarding grateloupia turuturu are disclosed in the article entitled, “Grateloupia Turuturu (Halymeniaceae, Rhodophyta) is the Correct Name of the Non-Native Species in the Atlantic Known as Grateloupia Doryphora,” Eur. J. Phycol. (2002), 37: 349-359, as authored by Brigitte Gavio and Suzanne Fredericq, the disclosure which is incorporated by reference in its entirety.

Porphyridium cruentum is a red algae in the family porphyridiophyceae and also termed rhodophyta and is used as a source for fatty acids, lipids, cell-wall polysaccharides and pigments. The polysaccharides of this species are sulphated. Some porphyridium cruentum biomass contains carbohydrates of up to 57%.

Monodus subterraneus is described in an article entitled, “Biosynthesis of Eicosapentaenoic Acid (EPA) in the Fresh Water Eustigmatophyte Monodus Subterraneus (Eustigmatophyceae),” J. Phycol, 38, 745-756 (2002, authored by Goldberg, Shayakhmetova, and Cohen, the disclosure which is incorporated by reference in its entirety. The biosynthesis of PUFAs from algae is complicated and the biosynthesis from this algae is described in that article.

Phaeodactylum tricornutum is a diatom and unlike most diatoms, it can grow in the absence of silicon and the biogenesis of silicified frustules is facultative.

Isochrysis galbana is a microalgae and used in the bivalve aquaculture industry.

Navicula sp. is a boat-shaped algae and is a diatom. Pythium irregule is a soilborne pathogen found on plant hosts.

Nannochioropsis sp. occurs in a marine environment, but also occurs in fresh and brackish water. The species are small, nonmotile spheres that do not express any distinct morphological feature. These algae have chlorophyll A and lack chlorophyll B and C. They can build high concentrations of pigment such as astaxanthin, zeaxanthin and canthaxinthin. They are about 2-3 micrometers in diameter. They may accumulate high levels of polyunsaturated fatty acids.

Nitzschia sp. is a pinnate marine diatom and usually found in colder waters and associated with both Arctic and Antarctic polar sea ice where it is a dominant diatom. It produces a neurotoxin known as domoic acid which is responsible for amnesic shell fish poisoning. It may grow exponentially at temperatures between −4 and −6 degrees C. It may be processed to form and extrapolate the fatty acids.

As a source of polyunsaturated fatty acids, microalgae competes with other micro-organisms such as fungi and bacteria. There may be some bacterial strains that could be an EPA source, but microalgae has been found to be a more adequate and readily available source. Microalgae is a good source of oil and EPA when derived from phaeodactylum, isochrysis and monodus. The microalgae phaeodactylum tricornutum produces a high proportion of EPA. Other different strains and species of microalgae, fungi and possibly bacteria that can be used to source EPA include the following:

### I. Diatoms

Asterionella japonica
Bidulphia sinensis
Chaetoceros septentrionale
Lauderia borealis
Navicula biskanteri
Navicula laevis (heterotrof.)
Navicula laevis
Navicula incerta
Stauroneis amphioxys
Navicula pellicuolsa
Bidulphia aurtia
Nitzschia alba
Nitzschia chosterium
Phaeodactylum tricornutum
Phaeodactylum tricornutum
Skeletonema costatum

### II. Chrysophyceae

Pseudopedinella sp.
Cricosphaera elongate

### III. Eustigmatophyceae

Monodus subterraneus
Nannochloropsis

### IV. Prymnesiophyceae

Rodela violacea 115.79
Porphyry. Cruentum 1380.Id

### V. Prasinophyceae

Pavlova salina

### VI. Dinophyceae

Cochlodinium heteroloblatum
Cryptecodinium cohnii
Gonyaulax catenella
Gyrodinium cohnii
Prorocentrum minimum

### VII. Other Microalgae

Chlorella minutissima
Isochrysis galbana ALII4
Phaeodactylum tricornutum WT
Porphyridium cruentum
Monodus subterraneus

### VIII. Fungi

Mortierella alpine
Mortierella alpine IS-4
Pythium irregulare

### IX. Bacteria

SCRC-2738

Different microalgae may be used to form the algae based oil comprising glycolipids and phospholipids and at least EPA and/or EPA/DHA. Examples include: Chlorophyta, Cyanophyta (Cyanobacteria), and Heterokontophyta. The microalgae may be from one of the following classes: Bacillariophyceae, Eustigmatophyceae, and Chrysophyceae. The microalgae may be from one of the following genera: Nannochloropsis, Chlorella, Dunaliella, Scenedesmus, Selenastrum, Oscillatoria, Phormidium, Spirulina, Amphora, and Ochromonas.

Other non-limiting examples of microalgae species that may be used include: Achnanthes orientalis, Agmenellum spp., Amphiprora hyaline, Amphora coffeiformis, Amphora coffeiformis var. linea, Amphora coffeiformis var. punctata, Amphora coffeiformis var. taylori, Amphora coffeiformis var. tenuis, Amphora delicatissima, Amphora delicatissima var. capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomas perigranulata, Chlorella anitrata, Chlorella antarctica, Chlorella aureoviridis, Chlorella candida, Chlorella capsulate, Chlorella desiccate, Chlorella ellipsoidea, Chlorella emersonii, Chlorella fusca, Chlorella fusca var. vacuolata, Chlorella glucotropha, Chlorella infusionum, Chlorella infusionum var. actophila, Chlorella infusionum var. auxenophila, Chlorella kessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebouxioides, Chlorella vulgaris, Chlorococcurn infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Effipsoidon sp., Euglena spp., Franceia sp., Fragilaria crotonensis, Fragilaria sp., Gleocapsa sp., Gloeothamnion sp., Haematococcus pluvialis, Hymenomonas sp., Isochrysis aff. galbana, Isochrysis galbana, Lepocinclis, Micractinium, Micractinium, Monoraphidium minutum, Monoraphidium sp., Nannochloris sp., Nannochloropsis salina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrine, Nitzschia closterium, Nitzschia communis, Nitzschia dissipate, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachiorella kessleri, Pascheria acidophila, Pavlova sp., Phaeodactylum tricomutum, Phagus, Phormidium, Platymonas sp., Pleurochrysis carterae, Pleurochrysis dentate, Pleurochrysis sp., Prototheca wickerhamii, Prototheca stagnora, Prototheca portoricensis, Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissflogii, and Viridiella fridericiana. Preferably, the microalgae are autotrophic.

It is also possible to form the oil comprising glycolipids and phospholipids and at least EPA from genetically modified yeast. Non-limiting examples of yeast that can be used include: Cryptococcus curvatus, Cryptococcus terricolus, Lipomyces starkeyi, Lipomyces lipofer, Endomycopsis vernalis, Rhodotorula glutinis, Rhodotorula gracilis, Candida 107, Saccharomyces paradoxus, Saccharomyces mikatae, Saccharomyces bayanus, Saccharomyces cerevisiae, any Cryptococcus, C. neoformans, C. bogoriensis, Yarrowia lipolytica, Apiotrichum curvatura, T. bombicola, T. apicola, T. petrophilum, C. tropicalis, C. lipolytica, and Candida albicans. It is even possible to use a biomass as a wild type or genetically modified fungus. Non-limiting examples of fungi that may be used include Mortierella, Mortierrla vinacea, Mortierella alpine, Pythium debaryanurn, Mucor circinelloides, Aspergillus ochraceus, Aspergillus terreus, Pennicillium iilacinum, Hensenulo, Chaetomium, Cladosporium, Malbranchea, Rhizopus, and Pythium.

It is also possible that bacteria may be used that includes lipids, proteins, and carbohydrates, whether naturally occurring or by genetic engineering. Non-limiting examples of bacteria include: Escherichia coli, Acinetobacter sp. any actinomycete, Mycobacterium tuberculosis, any streptomycete, Acinetobacter calcoaceticus, P. aeruginosa, Pseudomonas sp., R. erythropolis, N. erthopolis, Mycobacterium sp., B., U. zeae, U. maydis, B. lichenformis, S. marcescens, P. fluorescens, B. subtilis, B. brevis, B. polmyma, C. lepus, N. erthropolis, T. thiooxidans, D. polymorphis, P. aeruginosa and Rhodococcus opacus.

Possible algae sourced, EPA/DHA based oils that are derived from an algae and contain glycol and phospholipid bound EPA and/or EPA/DHA and may include a significant amount of free fatty acids, triglycerides and phospholipids and glycolipids in the range of 35-40% or more of total lipids are disclosed in the treatise “Chemicals from Microalgae” as edited by Zvi Cohen, CRC Press, 1999. Reference is also made to a study in men that have been given a single dose of oil from a polar-lipid rich oil from the algae nannochloropis oculata as a source of EPA and described in the article entitled, “Acute Appearance of Fatty Acids in Human Plasma—A Comparative Study Between Polar-Lipid Rich Oil from the Microalgae Nannochloropis Oculata in Krill Oil in Healthy Young Males,” as published in Lipids in Health and Disease, 2013, 12:102 by Kagan et al. The EPA in that algae oil was higher than that of krill oil by about 25.06 to 13.63 for fatty acid composition as the percent of oil. The algae oil was provided at 1.5 grams of EPA and no DHA as compared to krill oil that was provided at 1.02 grams EPA and 0.54 grams DHA. The participants consumed both oils in random order and separated by seven days and the blood samples were collected before breakfast and at several time points up to 10 hours after taking the oils.

The researchers determined that the algae based oil had a greater concentration of EPA and plasma than krill oil with the EPA concentration higher with the algae based oil at 5, 6, 8 and 10 hours (P<0.05) intended to be higher at 4 hours (P=0.094). The maximum concentration (CMAX) of EPA was higher with algae oil than with krill oil (P=0.010). The maximum change in concentration of EPA from its fasting concentration was higher than with krill oil (P=0.006). The area under the concentration curve (AUC) and the incremental AUC (IAUC) was greater (P=0.020 and P=0.006). This difference may relate to the different chemical composition and possibly the presence of the glycolipids where the presence of DHA in krill oil limits the incorporation of EPA into plasma lipids. Also, the n-3 polyunsaturated fatty acids within glycolipids as found in the algae oil, but not in a krill oil, may be an effective system for delivering EPA to humans.

The incorporated by reference '037 patent describes the benefit of using an algae based oil, and more particularly, a marine based algae oil and discloses different manufacturing and production techniques. Microalgae can be cultured photoautotrophically outdoors to prepare concentrated microalgae products containing Eicosapentaenoic acid (EPA) and Docosahexaenoic acid (DHA), which are the long-chain polyunsaturated fatty acids (PUFAs) found in fish oil. Both are very important for human and animal health. The concentrated microalgae products as disclosed in the '037 patent may contain EPA and DHA and lipid products containing EPA and DHA purified from microalgae. The concentrated microalgae composition may be prepared by cultivating microalgae photoautotrophically outdoors in open ponds under filtered sunlight in a continuous or batch mode and at a dilution rate of less than 35% per day. The microalgae may be harvested in the exponential phase when the cell number is increasing at a rate of at least 20% of maximal rate. In one example, the microalgae is concentrated. In another example, at least 40% by weight of lipids in the microalgae are in the form of glycodiacylglycerides, phosphodiacylglycerides, or a combination thereof and at least 5% by weight of the fatty acids are DHA, EPA, or a combination thereof.

In one example, the microalgae are Tetraselmis sp. cultivated at above 20° C. or in another example at above 30° C. The EPA yield in the microalgae has been found to be at least 10 mg/liter culture. The microalgae can be Isochrvsis sp. or Pavlova sp. in another example, or are Thalassiosira sp. or Chaetecoros sp. The microalgae may be different diatoms and are cultivated photoautotrophically outdoors in open ponds for at least 14 days under filtered sunlight and at least 20% by weight of the fatty acids are EPA.

The use of this algae based oil overcomes the technical problems associated with the dwindling supplies of fish oil and/or Antarctic krill, which are now more difficult to harvest and obtain and use economically because these products are in high demand. A major difference between fish oils and algae based oils is their structure. Fish oils are storage lipids and are in the form of triacylglycerides. The algae based oils as lipids are a mixture of storage lipids and membrane lipids. The EPA and DHA present in algae based oils are mainly in the form of glycolipids and a small percentage is in the form of phospholipids. Glycolipids are primarily part of chloroplast membranes and phospholipids are part of cell membranes.

The incorporated by reference '037 patent describes various methods for culturing microalgae photoautotrophically outdoors to produce EPA and DHA. One method used is filtering sunlight to reduce the light intensity on the photoautotrophic culture. Shade cloth or netting can be used for this purpose. It was determined that for most strains, the optimal solar intensity for growth, for maintaining a pure culture, and for omega-3 fatty acid accumulation was about 40,000 to 50,000 lux, approximately half of the 110,000 lux of full sunlight. Shade cloth or netting is suitable for filtering the sunlight to the desired intensity.

It is also possible to culture microalgae photoautotrophically outdoors and produce EPA and DHA by using small dilutions and a slow dilution rate of less than 40% per day, preferably less than 35% per day, more preferably from about 15% to about 30% per day. In other examples, the dilution rate is 15-40% per day or 15-35% per day, and in yet other examples, the dilution rate is 10-30%, 10-35%, or 10-40% per day. These smaller dilutions and lower dilution rates than are usually used help prevent contamination in outdoor photoautotrophic cultures. It also promotes thick culture growth that gives good DHA or EPA yield.

Another technique to successfully culture microalgae photoautotrophically outdoors and produce EPA and EPA/DHA is to harvest the microalgae in exponential phase rather than stationary phase. Harvesting in exponential phase reduces the risk of contamination in outdoor photoautotrophic cultures and has surprisingly been found to give a good yield of EPA and DHA. To drive fat accumulation in microbial cultures, the cultures are harvested in stationary phase because cells in the stationary phase tend to accumulate storage lipids. The '037 patent teaches that EPA and DHA accumulate in large amounts as membrane lipids in cultures harvested in the exponential phase. The membrane lipids containing EPA and DHA are predominantly phosphodiacylglycerides and glycodiacylglycerides, rather than the triaclyglycerides found in storage lipids. These cultures are harvested often when cell number is increasing at a rate at least 20% of the maximal rate, i.e., the maximal rate achieved at any stage during the outdoor photoautotrophic growth of the harvested culture. In specific examples, the cultures are harvested in exponential phase when cell number is increasing at a rate of at least 30%, at least 40%, or at least 50% of maximal rate. It is also possible to use recombinant DNA techniques.

The incorporated by reference '037 Patent includes several examples, which are referenced to the reader for description and teaching purposes as Examples 1-6.

This composition for eye or cardiovascular health possibly may be used for treatments besides eye care either as it is or with other added components. Some of these components are described above and others can be determined and added. The composition may include the phospholipid and seed oil extract and carotenoids and/or lutein and/or coenzyme Q10 and other oils such as from algae.

Possible uses of the composition include use as a treatment for depression that may counter neurological disorders associated with depression. This could include treatment for a deficiency of neurotransmitters at post-synaptic receptor sites. The composition may be used to treat manic episodes in bipolar treatments and treat panic disorder and reduce the frequency and severity of panic attacks and the severity of agoraphobia. The composition may be used to treat Obsessive Compulsive Disorder (OCD) and malfunctioning neurotransmitters and serotonin receptors. The composition may also be used in the treatment of Alzheimer's Disease (AD) and reduce the presence of aluminosilicates at the core of senile plaque and diseased neurons. The composition may be used to treat aging disorders for cellular differentiation, proliferation and regeneration. It may also be used to treat age-related changes in mitochondrial function and age-related hearing loss. The composition may also possibly maintain metabolic activity and available energy by maintaining levels of phospholipids in normal cells and maintain membrane integrity and regulate enzyme activities and membrane transport processes through changes in membrane fluidity.

The composition may be beneficial for biological functions of essential fatty acids, including neural tissues such as the brain and retina and treat dementia-related diseases to increase mental function, memory, concentration and judgment and overcome the effects of Alzheimer's Disease. The composition may also be used to restore and preserve liver function and protect the liver against damage from alcoholism, pharmaceuticals, pollutant substances, viruses and other toxic influences that may damage cell membranes. It may possibly have antioxidant activity.

Additives may be used with the composition and pharmaceutical or nutraceutical formulations may be made by methods known in the art. For example, the composition may be formulated in a conventional manner using one or more pharmaceutically or nutraceutically acceptable carriers. Thus, the composition may be formulated for oral administration. For oral administration, the pharmaceutical or nutraceutical compositions as compositions may take the form of, for example, tablets or capsules prepared by conventional techniques with pharmaceutically or nutraceutically acceptable excipients such as binding agents (e.g., pregelatinised maize starch, polyvinylpyrrolidone or hydroxypropyl methylcellulose); filters (e.g., lactose, microcrystalline cellulose or calcium phosphate); lubricants (e.g., magnesium stearate, talc or silica); disintegrants (e.g., potato starch or sodium starch glycollate); or wetting agents (e.g., sodium lauryl sulphate). The tablets may be coated by methods known in the art. Liquid preparations for oral administration may take the form of, for example, solutions, syrups or suspensions, or they may be presented as a dry product for use with water or another suitable vehicle before use. Such liquid preparations may be prepared by conventional techniques with pharmaceutically or nutraceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

When the composition is used as a nutraceutical, it can be in the form of foods, beverages, energy bars, sports drinks, supplements or other forms as known in the art. This composition is also useful in cosmetic preparations, e.g., moisturizing creams, sun-block products and other topical cosmetic products as known in the art.

The composition may possibly be used in the treatment or prevention of a variety of disorders or disease states including: liver disorder or disease; chronic hepatitis; steatosis; liver fibrosis; alcoholism; malnutrition; chronic parenteral nutrition; phospholipid deficiency; lipid peroxidation; disarrhythmia of cell regeneration; destabilization of cell membranes; coronary artery disease caused by hypercholesterolemia; high blood pressure; menopausal or post-menopausal conditions; cancer, e.g., skin cancer; hypertension; aging; benign prostatic hyperplasia; kidney disease; edema; skin diseases; gastrointestinal diseases; peripheral vascular system diseases (e.g. leg ulcers); pregnancy toxemia; and neurodegenerative and psychiatric diseases (e.g. Parkinson's, Alzheimer's, autism, attention deficit disorder, learning disorders, mood disorders, bipolar depression, multiple sclerosis, muscular dystrophy).

The composition may also be useful for targeting tumors and may be used in conjunction with radioisotopes for diagnosing central nervous system tumors. The composition may also be used to reduce local fat deposits and reducing visible cellulite. The composition may also be used in aesthetics such as breast enlargement by acting on the lobular tissue of the breast and by increasing hydration of the breast.

The composition may be used to treat and/or prevent cardiovascular disorders and diseases, arthritis, skin cancer, diabetes, premenstrual syndrome and transdermal transport enhancement. It may be used to decrease cholesterol in vivo and inhibit platelet adhesion and plaque formation and reduce vascular endothelial inflammation in a patient and offer hypertension prophylaxis. The composition may prevent oxidation of low-density lipoprotein and have an inhibitory effect on the secretion of VLDL possibly due to increased intracellular degradation of APO B-100. It may offer a post-myocardial infarction prophylaxis possibly because of its ability to decrease CIII apolipoprotein B, to decrease C3 non-apoliproprotein B lipoproteins and to increase antithrombin 3 levels. It may be suitable for prophylactic usage against cardiovascular disease in humans where it relates to coronary disease, hyperlipidemia, hypertension, ischemic disease such as relating to angina, myocardial infarction, cerebral ischemia, and shock without clinical or laboratory evidence of ischemia or arrhythmia.

The composition may be suitable to offer symptomatic relief for arthritis, Still's Disease, polyarticular or pauciarticular juvenile rheumatoid arthritis, rheumatoid arthritis, osteoarthritis, and may provide clinical improvement in decreasing the number of tender joints and analgesics consumed daily by decreasing the production of interleukin and interleukin-1 in human patients. The composition may also be used as a skin cancer prophylactic. It may have some retinal and anti-carcinogenic effects. It may enhance transdermal transportation as a substrate for dermatological topical therapeutic applications and may be used in dermatological treatments via creams, ointments, gels, lotions and oils and may be used in various therapeutic applications such as relating to anesthesic, corticosteroids, anti-inflammatory, antibiotic and ketolytic functions. It may also be used to enhance transdermal transportation as a substrate for dermatological topical cosmetic applications where cosmetic application relates to skin hydration, anti-wrinkle, caratolytics, peeling and mask via creams, ointments, gels, lotions or oils. The composition may be used to reduce the pain and mood changes associated with premenstrual syndrome in women.

The composition may be used to treat or prevent a cardiometabolic disorder/metabolic syndrome. The cardiometabolic disorder could be atherosclerosis, arteriosclerosis, coronary heart (carotid artery) disease (CHD or CAD), acute coronary syndrome (or ACS), valvular heart disease, aortic and mitral valve disorders, arrhythmia/atrial fibrillation, cardiomyopathy and heart failure, angina pectoris, acute myocardial infarction (or AMI), hypertension, orthostatic hypotension, shock, embolism (pulmonary and venous), endocarditis, diseases of arteries, the aorta and its branches, disorders of the peripheral vascular system (peripheral arterial disease or PAD), Kawasaki disease, congenital heart disease (cardiovascular defects) and stroke (cerebrovascular disease), dyslipidemia, hypertriglyceridemia, hypertension, heart failure, cardiac arrhythmias, low HDL levels, high LDL levels, stable angina, coronary heart disease, acute myocardial infarction, secondary prevention of myocardial infarction, cardiomyopathy, endocarditis, type 2 diabetes, insulin resistance, impaired glucose tolerance, hypercholesterolemia, stroke, hyperlipidemia, hyperlipoproteinemia, chronic kidney disease, intermittent claudication, hyperphosphatemia, omega-3 deficiency, phospholipid deficiency, carotid atherosclerosis, peripheral arterial disease, diabetic nephropathy, hypercholesterolemia in HIV infection, acute coronary syndrome (ACS), non-alcoholic fatty liver disease/non-alcoholic steatohepatitis (NAFLD/NASH), arterial occlusive diseases, cerebral atherosclerosis, arteriosclerosis, cerebrovascular disorders, myocardial ischemia, coagulopathies leading to thrombus formation in a vessel and diabetic autonomic neuropathy.

The composition may also be used to treat, prevent or improve cognition and/or a cognitive disease, disorder or impairment (memory, concentration, learning (deficit)), or of treating or preventing neurodegenerative disorders. The cognitive disease, disorder or impairment could be Attention Deficit Disorder (ADD), Attention Deficit Hyperactivity Disorder (ADHD), autism/autism spectrum disorder (ASD), (dyslexia, age-associated memory impairment and learning disorders, amnesia, mild cognitive impairment, cognitively impaired non-demented, pre-Alzheimer's disease, Alzheimer's disease, epilepsy, Pick's disease, Huntington's disease, Parkinson disease, Lou Gehrig's disease, pre-dementia syndrome, Lewy body dementia dementia, dentatorubropallidoluysian atrophy, Freidreich's ataxia, multiple system atrophy, types 1, 2, 3, 6, 7 spinocerebellar ataxia, amyotrophic lateral sclerosis, familial spastic paraparesis, spinal muscular atrophy, spinal and bulbar muscular atrophy, age-related cognitive decline, cognitive deterioration, moderate mental impairment, mental deterioration as a result of ageing, conditions that influence the intensity of brain waves and/or brain glucose utilization, stress, anxiety, concentration and attention impairment, mood deterioration, general cognitive and mental well-being, neurodevelopmental, neurodegenerative disorders, hormonal disorders, neurological imbalance or any combinations thereof. The cognitive disorder may be memory impairment.

The composition may be used to inhibit, prevent or treat inflammation or an inflammatory disease. The inflammation or inflammatory disease may be due to organ transplant rejection; reoxygenation injury resulting from organ transplantation (see Grupp et al., J. Mol. Cell. Cardiol. 31: 297-303 (1999)) including, but not limited to, transplantation of the following organs: heart, lung, liver and kidney; chronic inflammatory diseases of the joints, including arthritis, rheumatoid arthritis, osteoarthritis and bone diseases associated with increased bone resorption; inflammatory bowel diseases (IBD) such as ileitis, ulcerative colitis (UC), Barrett's syndrome, and Crohn's disease (CD); inflammatory lung diseases such as asthma, acute respiratory distress syndrome (ARDS), and chronic obstructive pulmonary disease (COPD); inflammatory diseases of the eye including corneal dystrophy, trachoma, onchocerciasis, uveitis, sympathetic ophthalmitis and endophthalmitis; chronic inflammatory diseases of the gum, including gingivitis and periodontitis; inflammatory diseases of the kidney including uremic complications, glomerulonephritis and nephrosis; inflammatory diseases of the skin including sclerodermatitis, psoriasis and eczema; inflammatory diseases of the central nervous system, including chronic demyelinating diseases of the nervous system, multiple sclerosis, AIDS-related neurodegeneration and Alzheimer's disease, infectious meningitis, encephalomyelitis, Parkinson's disease, Huntington's disease, epilepsy, amyotrophic lateral sclerosis and viral or autoimmune encephalitis, preeclampsia; chronic liver failure, brain and spinal cord trauma, and cancer. The inflammatory disease may also be a systemic inflammation of the body, exemplified by gram-positive or gram negative shock, hemorrhagic or anaphylactic shock, or shock induced by cancer chemotherapy in response to pro-inflammatory cytokines, e.g., shock associated with pro-inflammatory cytokines. Such shock can be induced, e.g., by a chemotherapeutic agent that is administered as a treatment for cancer. Other disorders include depression, obesity, allergic diseases, acute cardiovascular events, muscle wasting diseases, and cancer cachexia. Also, inflammation that results from surgery and trauma may possibly be treated.

Some proposals as supplements for eye health have used specific ratios of lutein and zeaxanthin, such as a 5:1 ratio of lutein to zeaxanthin with a beadlet delivery technology to protect the carotenoids and provide greater stability during manufacturing. This use of lutein and zeaxanthin has been found advantageous in blue light protection, which is important when some users are in constant use of computer screens. Some of these compositions also use a specific ratio of zeaxanthin isomers (R,R- and R,S[meso]-zeaxanthin) at a 5:1 ratio where the lutein is dominant. Astaxanthin and a carrier such as including a lipid or fatty acid or combination is not used in many of these formulations and not suggested, since the carotenoids lutein and zeaxanthin are normally found in the eyes, but astaxanthin is not.

Another supplement proposal uses a therapeutically effective amount of ocular antioxidants such as carotenoids and includes antioxidant minerals and extracts, including as essential ingredients lutein, zeaxanthin, beta-carotene, retinoids, retinal, retinaldehyde and meso-zeaxanthin. That formula may optionally include other minerals and ingredients. This supplement has been found advantageous to treat Age-Related Macular Degeneration in some sufferers, but it has not been shown to treat other disorders, specifically, any associated with ocular fatigue and eye focusing in humans in a manner as efficiently and advantageously as the current formulation from the inventors having the carrier that may include the lipid or fatty acid and the three specific carotenoids in their ranges, and more specifically, the astaxanthin, lutein and zeaxanthin, which also have specific functions that interoperate together and function in a specific wavelength absorbance. The inclusion of other carotenoids may interfere with the performance and effectiveness of the astaxanthin, lutein and zeaxanthin in their ranges.

The inventors have determined that the carrier, which includes in a specific example the lipid, fatty acid, or combination, admixed with carotenoids such as 0.5 to 8 mg of astaxanthin, 2.0 to 15 mg of lutein, and 0.2 to 12 mg of zeaxanthin and formulated into an oral dosage form as a single dosage capsule, is effective when administered in a therapeutic amount as a dietary supplement composition to treat photo-induced ocular fatigue and associated reduction in speed of ocular focus in humans. It has been found even more advantageous when the carrier is at least one of a lipid and a fatty acid so that selected lipids alone or in combination with fatty acids may be used. In one example, 50 to 700 mg of the lipid, fatty acid or a combination may be admixed with carotenoids that in a preferred example has been found to have remarkable efficacy at 4.0 to 6.0 mg of astaxanthin especially that derived from Haematococcus pluvialis (Hp), 10 to 12 mg of lutein, and 1.0 to 2.5 mg of zeaxanthin, and formulated into an oral dosage form as a single dosage capsule.

Based upon this research and coupled with previous determinations discussed above, it is possible to administer a therapeutic amount of a dietary supplement composition that includes 50 to 700 mg of at least one of a carrier that includes the lipid and the fatty acid or combination of both, admixed with astaxanthin, lutein and zeaxanthin in the specific ranges. This dietary supplement composition is formulated into a single dosage capsule, and in an example, the astaxanthin is about 0.1 to 16% by weight of the carrier that may include at least one of the lipid and fatty acid, and the lutein is about 0.4 to 30% by weight of the carrier that may include at least one of the lipid and fatty acid, and the carrier that includes, in an example, trans-zeaxanthin that is about 0.04 to 24% by weight of the carrier that may include at least one of the lipid and fatty acid.

The carrier may be a lipid and formed as phospholipids that are 5% to 60% by weight of the composition and may also be formed as a seed based phospholipid such as from a sunflower, canola, soybean, and other seed based sources, some of which are described above. Phospholipids may also be derived from a marine source such as krill oil or some fish oils. The composition may include a seed oil containing phospholipid bound EPA and DMA as described above, and of course, the fatty acid may be formed as an omega-3 fatty acid, which often is preferred in some cases depending on the particular user requirements. Although a range of 50 to 700 mg is disclosed as optimal for a carrier including a lipid, fatty acid or combination, another optimal range is 300 to 500 mg of the carrier such as the lipid or fatty acid or a combination. Many different lipids and fatty acids are described above and can be included in different seed oils, marine oils, and similar types of carriers.

The range of the carrier in a single dosage capsule may vary, but usually is about 500 mg and can vary usually from 50 mg to 700 mg on the high end, such as when users or manufacturers may request more omega-3's as compared to lower ranges that may be used if greater phospholipids are utilized. For example, the carrier, including lipid or a fatty acid or combination, could be 50 to 500 mg or 300 to 500 mg. The carotenoids can vary in a broad range from 0.5 to 8.0 mg of astaxanthin, 2.0 to 15.0 mg of lutein, and 0.2 to 12.0 mg of zeaxanthin, to a preferred range used in a specific formulation found advantageous of 4.0 to 6.0 mg of astaxanthin derived from Haematococcus pluvialis, 10 to 12 mg of lutein, and 1.0 to 2.5 mg of zeaxanthin. Other ranges for the astaxanthin could be 0.5 to sometimes 12.0 mg, 1.0 to 10.0 mg, 2 to 10 mg, 4.0 to 8.0 mg, and other ranges.

The lutein can vary from 4.0 to 14.0 mg, or 6.0 to 12.0 mg and other ranges. The zeaxanthin in an example may be 10% to 20% of the lutein ranges in an example, and 10% to 20% of any specific amount of lutein. The zeaxanthin may include meso-zeaxanthin and (R,R) zeaxanthin or other isomers, but those in some cases may be preferred. The ratio of lutein and zeaxanthin isomers may vary and could be in the range of about 4:1 to about 6:1 in some non-limiting examples. The lutein could be trans-lutein and the zeaxanthin could be (R,R)-zeaxanthin and some (R,S)-zeaxanthin.

This use of the composition as described helps a human with eye accommodation, wherein the eye changes optical power to maintain a clear image of focus on an object as its distance varies. The formulation also helps in blue light filtration. This is important in many cases where users look at computer screens all day. Accommodation is similar to a reflex, but can also be consciously controlled where optical power can be varied using the ciliary body up to about 15 diopters in humans as a result of the reduction in ocular tension induced by ciliary muscle contraction that can occur in some cases in as little as 350 milliseconds. With the increased usage of modern computer screens, individuals sometimes find that their ability to accommodate and adjust for photo-induced ocular fatigue and associated reduction in the speed of ocular focus requires supplementation to help. The inventors have determined that the formulation using the carrier that may include the lipid, fatty acid or combination, and the three carotenoids of astaxanthin, lutein, and zeaxanthin with their preferred ranges as described is advantageous to help alleviate and treat photo-induced ocular fatigue and associated reduction in speed of ocular focus in humans.

Some research has been accomplished on using lutein and astaxanthin in eye health formulas, but that research has been combined with additional components such as cyanidin-3-glucoside and Docosahexaenoic Acid (DHA) in conjunction with Bilberry Extract and Black Soybean Hull Extract. Although that composition had some effect to improve accommodative ability and symptoms related to eye fatigue, that composition still required those specific and additional components, and did not include zeaxanthin, a component that the inventors of the current formulation found advantageous.

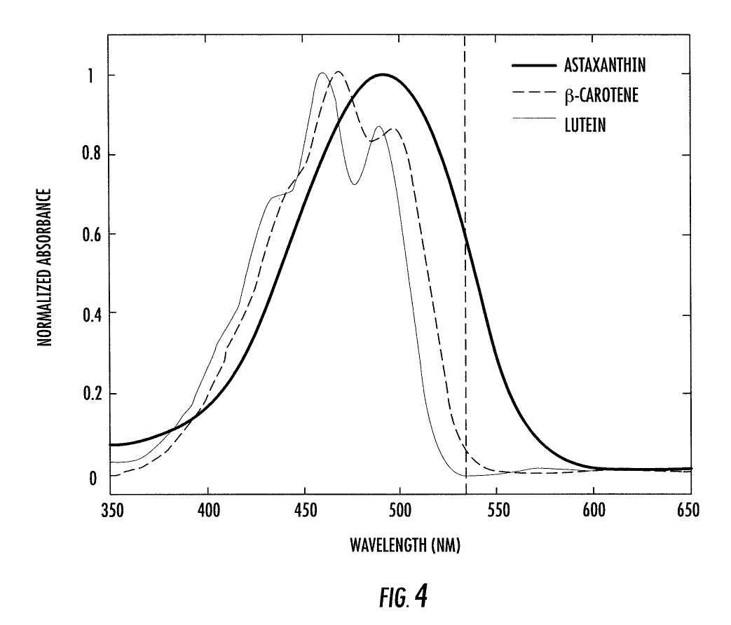

Referring now to FIG. 4, there is illustrated a graph from Collins, Carotenoid Distribution in Living Cells of Haematococcus pluvialis (Chlorophyceae) 2011, and showing the normalized absorbance for three carotenoids of astaxanthin, β-carotene, and lutein and showing the wavelength separation between astaxanthin and lutein. Lutein is just below 450 nanometers (nm) and astaxanthin is just below 500 nanometers. Zeaxanthin, on the other hand, but not illustrated in this graph, has been found closer to 480 or 490 nanometers, thus showing that the three carotenoids used in the current formulation having the astaxanthin, lutein and zeaxanthin are separated in nanometer wavelength for their absorption coefficients but operate together, each contributing to the overall efficacy of the composition, especially in their composition ranges as disclosed and with their carrier, for example, including the lipid or fatty acid, and to be advantageous to treat photo-induced ocular fatigue and associated reduction in speed of ocular focus in humans.

It has been found that a lutein monolayer with a phospholipid results in a blue-shift of the absorption bands as compared to pure carotenoid monolayer and macular pigment spectra. This may be advantageous when coupled with the carrier that includes the phospholipids and coupled in association with the astaxanthin, made more bioavailable by the phospholipids, and the zeaxanthin, especially with their specific absorption bands and when used in their specific concentration ranges.

It has been shown that astaxanthin, like lutein and zeaxanthin, may bind to the transport protein (XBP) as an xanthophyll binding protein and that astaxanthin may attenuate selenite-induced cataractogenesis in rats in vivo. Astaxanthin has been shown to protect porcine lens crystallins (lens protein) from oxidative damage by calcium ions activated protease (calpain) or iron mediated hydroxyl radicals in vitro. Some Japanese researchers have demonstrated that intravenous administration of astaxanthin suppressed rat paw injected LPS induced uveitis in rats in vivo and down regulated the pro-inflammatory factors NO, TNF-alpflexuron, PTET and NF-KB so that astaxanthin reduced the endotoxin induced ocular inflammation.

It has also been found that 2 mg/day of oral astaxanthin even over a minimal four week period has been found to reduce the measurable endogenous levels of DMA damage by 40% in some humans and as much as 6 mg/day dosage of astaxanthin has increased retinal capillary blood flow following four weeks of supplementation and reduced eye fatigue (asthenopia) and the rate of accommodation contraction time by 54% in humans (N=48). Some researchers have evaluated the effects of 6 mg/day of astaxanthin pretreatment for two weeks on superoxide scavenging activity and the levels of hydrogen peroxide and total hydroperoxides in human vitreous humor taken during separated untreated and astaxanthin treated bilateral cataract surgery patients (N=35). The superoxide scavenging activity in vitreous humor was significantly elevated and the total hydroperoxide levels were significantly lowered. This suggested that since astaxanthin intake clearly demonstrated enhanced superoxide scavenging activity and suppressed the total hydroperoxide production in human vitreous humor, it indicated the possibility that astaxanthin had suppressive effects on various oxidative stress-related diseases and showed the importance of human oral astaxanthin therapy such that astaxanthin gets into the human eye within two weeks of oral administration and does suppress oxidative damage in humans. Thus, orally administered astaxanthin will accumulate in humans in the iris-ciliary body and accumulate in the vitreous humor and is actively transported into the eye and likely accumulates on the human retinal macula, and thus, will have ocular anti-inflammatory effects and protection from oxidative damage by quenching free radicals and superoxide and reduce the ocular exposure to damaging blue light through the broad spectral absorption and improve the accommodation contraction time in the visual display terminal eye fatigue and prevent DHA damage.

It should be understood that the retina is a highly vascularized tissue structure providing the highest oxygen tension of any human tissue and high energy light impingement induces damaging free radicals in singlet oxygen formation. The macular carotenoids may absorb light in the blue-green region of the visible spectrum and may act as high energy filters and prevent cellular damage by quenching free radicals and/or singlet oxygen, thus suppressing photo-oxidative damage. The body does not produce carotenoids, and thus, the dietary intake of certain carotenoid xanthophylls is essential to overall eye health. Astaxanthin does accumulate in the human vitreous humor where it increases the superoxide scavenging activity while lowering overall total hydroperoxide formation. Different human studies conducted in Italy and Japan indicate that astaxanthin provides favorable relief for VDT workers via faster accommodative contraction and reduced asthenopia with faster fatigue recovery, and thus, the astaxanthin is a complement to the lutein and zeaxanthin and especially at the concentrations as claimed with the carrier to address the photo-induced ocular fatigue and associated reduction in speed of ocular focus in humans.

The composition may also protect and contribute to normal macular pigment deposits and is a potent down regulator of ocular inflammatory cytokine expression. One test product as described in the article entitled, “Carotenoids in Age-Related Maculopathy Italian Study (CARMIS): Two-Year Results of a Randomized Study,” European Journal of Ophthalmology (2011) indicates that higher antioxidant intakes can prevent some age-related macular degeneration (AMD) when using carotenoids with other ingredients, including vitamin C (180 mg), vitamin E (30 mg), zinc (22.5 mg), copper (1 mg), lutein (10 mg), zeaxanthin (1 mg), and astaxanthin (4 mg). Although lutein, zeaxanthin and astaxanthin were used, they were also used in a required combination with specific ingredients, including vitamins C and E. The inventors of the current formulation have found that such supplementation, i.e., vitamins C and E, are not necessary. Indeed, use of the three carotenoids of the current formulation have been used in combination with krill oil and found effective and the effectiveness had also been found with other carriers, including the lipid and fatty acids, without requiring the addition of these other ingredients as described in the Italian study referenced above.

The formulation has also been found advantageous for age-related macular degeneration where the largest irreversible cause of vision loss is in the elderly at 6.5% of the world's population greater than 50 years old, who have various levels of ARMD, including 7.3 million U.S. citizens and 1.8 million U.S. citizens with advanced wet ARMD in at least one eye, according to a 2004 estimate. By 2020, there are some estimates that 3 million people will have wet ARMD based on aging demographics in the U.S. and 10% of dry (atrophic) ARMD patients may continue to degrade to a wet (exudative) ARMD diagnosis, thus resulting in eventual total vision loss for these people. There are some risk factors such as a genetic predisposition that includes a common single gene variant of complement factor H, which is associated with pathogenesis to ARMD, with 50% of the ARMD patients having a strong age-dependence likely attributed to losses in critical DNA expression because of ROS driven strand breaks and age-related telomere loss and the large soft drusen formation on the macula that increases the risk of developing ARMD.

Cigarette smoking and previous cataract surgery and familial history may have an impact as well as excess light exposure because the retina is highly vascularized neural tissue that has a high rate of metabolism and cellular turnover and because it has the highest consumption of oxygen per unit weight of any other tissue and is highly susceptible to light-induced ROS formation and resulting ROS-induced damage. Also dry ARMD is a prerequisite for wet ARMD because retinas with uncontrolled choroidal neovascularization tend to lead to total visual loss when the VEGF is greater than the PED (pigment epithelium derived factor) and goes out of homeostasis.

Any impairment of the retinal pigmented epithelium (RPE) cell function is an early and crucial event leading to clinically relevant ARMD, which is responsible for maintenance of the blood retinal barrier and the visual rhodopsin cycle and phagocytic uptake and degradation of constantly shed apical photoreceptor outer segments as cellular death, clean-up and regeneration. The RPE impairment leads to progressive accumulation of lipofuscin granules that are composed of lipids and proteins derived from phagosomal and/or lysosomal conversion of damaged photoreceptor proteins and progressive drusen formation. Lipofuscin accumulates in the RPE and is cytotoxic because it is a potent photo-inducible generator of ROS, which damages proteins, lipids and DNA. The drusen is an amorphous protein composite that includes apolipoproteins and cellular debris from the complement immune system and it deposits between the RPE and the bruch's membrane at the choriocapillaris.

Thus, proteomic and immunohistochemical analysis of drusen reveals many immune associated elements that include the complement cascade with uncontrolled gene expression associated with cell death. Other tests have shown that lutein and zeaxanthin, including an omega-3 fish oil polyunsaturated fatty acid supplement (DHA/EPA) did not affect the progression of ARMD while the AREDS1 study showed that some antioxidant vitamins help. An association of beta-carotene supplementation of lung cancer showed that it is possible to exchange beta-carotene with the mixture of lutein C and zeaxanthin and naturally occurring macular xanthophylls is beneficial.

Although some of these studies show the benefits of lutein and zeaxanthin, the inventors of the current formulation have found that the use of the three components in the ranges for the astaxanthin, lutein and zeaxanthin complement each other and are useful for the dietary supplement composition when used with a carrier that may include the lipid or fatty acid or their combination and the concentrations and ranges have been found advantageous, including the advantages of the specific normalized wavelength absorption ranges.

Many modifications and other embodiments of the invention will come to the mind of one skilled in the art having the benefit of the teachings presented in the foregoing descriptions and the associated drawings. Therefore, it is to be understood that the invention is not to be limited to the specific embodiments disclosed, and that the modifications and embodiments are intended to be included within the scope of the dependent claims.

That which is claimed is:

1. A method of treating photo-induced ocular fatigue and associated reduction in speed of ocular focus in humans, which comprises administering a therapeutic amount of a dietary supplement composition comprising a carrier admixed with carotenoids consisting of 0.5 to 8.0 mg of astaxanthin, 2.0 to 15 mg of lutein and 0.2 to 12.0 mg of zeaxanthin, and wherein the composition is formulated into an oral dosage form as a single dosage capsule.

2. The method according to claim 1 wherein said carrier comprises at least one of a lipid and a fatty acid.

3. The method according to claim 2 wherein said lipid comprises phospholipids that are 5% to 60% by weight of the composition.

4. The method according to claim 3 wherein said phospholipids comprise seed based phospholipids.

5. The method according to claim 3 wherein said phospholipids comprise phospholipids derived from a marine source.

6. The method according to claim 2 further comprising a seed oil containing phospholipid bound EPA and DHA.

7. The method according to claim 2 wherein said fatty acid comprises an omega-3 fatty acid.

8. The method according to claim 2 wherein the carotenoids consist of 4.0 to 6.0 mg of astaxanthin derived from Haematococcus pluvialis (Hp), 10 to 12 mg of lutein and 1.0 to 2.5 mg of zeaxanthin.

9. A method of treating photo-induced ocular fatigue and associated reduction in speed of ocular focus in humans, which comprises administering a therapeutic amount of a dietary supplement composition comprising 50 to 700 mg of a carrier, including at least one of a lipid and a fatty acid and admixed with carotenoids comprising 4.0 to 6.0 mg of astaxanthin derived from Haematococcus pluvialis (Hp), 10 to 12 mg of lutein and 1.0 to 2.5 mg of zeaxanthin, and wherein the composition is formulated into an oral dosage form as a single dosage capsule.

10. The method according to claim 9 wherein said lipid comprises phospholipids that are 5% to 60% by weight of the composition.

11. The method according to claim 10 wherein said phospholipids comprise seed based phospholipids.

12. The method according to claim 10 wherein said phospholipids comprise phospholipids derived from a marine source.

13. The method according to claim 9 further comprising a seed oil containing phospholipid bound EPA and DHA.

14. The method according to claim 9 wherein said fatty acid comprises an omega-3 fatty acid.

15. A method of treating photo-induced ocular fatigue and associated reduction in speed of ocular focus in humans, which comprises administering a therapeutic amount of a dietary supplement composition comprising 50 to 700 mg of a carrier, including at least one of a lipid and a fatty acid, and admixed with astaxanthin, lutein and trans-zeaxanthin, wherein the dietary supplement composition is formulated into a single dosage capsule, and wherein the astaxanthin is about 0.1 to 16, percent by weight of the at least one of the lipid and fatty acid, the lutein is about 0.4 to 30 percent by weight of the at least one of the lipid and fatty acid, and the trans-zeaxanthin is about 0.04 to 24 percent by weight of the carrier, including at least one of the lipid and fatty acid.

16. The method according to claim 15 wherein the composition comprises 0.5 to 8 mg of astaxanthin, 2 to 15 mg of lutein and 0.2 to 12 mg of trans-zeaxanthin.

17. The method according to claim 15 wherein the composition comprises about 4 to 6 mg of astaxanthin, about 10 to 12 mg of lutein and about 1.0 to 2.5 mg of trans-zeaxanthin.

18. The method according to claim 15 wherein the lipid comprises a seed based phospholipid.

19. The method according to claim 15 wherein said fatty acid comprises an omega-3 fatty acid.
